(19) United States
(12) Patent Application Publication
(10) Pub. No.: US 2022/0202956 A1
(43) Pub. Date: Jun. 30, 2022

(54) ADENO-ASSOCIATED VIRUS DELIVERY OF CLN6 POLYNUCLEOTIDE

(71) Applicant: RESEARCH INSTITUTE AT NATIONWIDE CHILDREN'S HOSPITAL, Columnbus, OH (US)

(72) Inventors: Kathrin Meyer, Columbus, OH (US); Brian K. Kaspar, Columbus, OH (US)

(21) Appl. No.: 17/426,993

(22) PCT Filed: Feb. 4, 2020
(86) PCT No.: PCT/US2020/016541

§ 371 Date: Jul. 29, 2021

Related U.S. Application Data
(60) Provisional application No. 62/923,125, filed on Oct. 18, 2019.
(60) Provisional application No. 62/912,977, filed on Oct. 9, 2019.
(60) Provisional application No. 62/881,151, filed on Jul. 31, 2019.
(60) Provisional application No. 62/880,641, filed on Jul. 30, 2019.
(60) Provisional application No. 62/800,915, filed on Feb. 4, 2019.

(51) Int. Cl. A61K 48/00; C12N 15/86; C07K 14/47; A61P 25/00

(52) U.S. Cl. CPC A61K 48/005; C12N 15/86; C12N 2750/14143; A61P 25/00; C07K 14/47

(57) ABSTRACT

The present disclosure relates to recombinant adeno-associated virus (rAAV) delivery of a neuronal ceroid lipofuscinosis neuronal 6 (CLN6) polynucleotide. The disclosure provides rAAV and methods of using the rAAV for CLN6 gene therapy of the neuronal ceroid lipofuscinosis or CLN6-Batten Disease.

This application claims priority benefit of U.S. Provisional Patent Application No. 62/800,915, filed Feb. 4, 2019, U.S. Provisional Patent Application No. 62/880,641, filed Jul. 30, 2019, U.S. Provisional Patent Application No. 62/881,151, filed Jul. 31, 2019, U.S. Provisional Patent Application No. 62/912,977, filed Oct. 9, 2019, and U.S. Provisional Patent Application No. 62/923,125, filed Oct. 18, 2019, all of these applications are incorporated herein by reference in their entirety.

### INCORPORATION BY REFERENCE OF THE SEQUENCE LISTING

This application contains, as a separate part of disclosure, a Sequence Listing in computer-readable form (filename: 53894_SeqListing.txt; 24,923 bytes—ASCII text file created Jan. 31, 2020) which is incorporated by reference herein in its entirety.

### FIELD

The present disclosure relates to recombinant adeno-associated virus (rAAV) delivery of a ceroid lipofuscinosis neuronal 6 (CLN6) polynucleotide. The disclosure provides rAAV and methods of using the rAAV for CLN6 gene therapy of the neuronal ceroid lipofuscinosis (NCL) or CLN6-Batten Disease.

### BACKGROUND

Neuronal ceroid lipofuscinoses (NCLs) are a group of severe neurodegenerative disorders, which are collectively referred to as Batten disease. These disorders affect the nervous system and typically cause worsening problems with e.g. movement and thinking ability. The different NCLs are distinguished by their genetic cause.

CLN6-Batten disease can occur as two different forms: variant late-infantile (vLINCL), the more common form, and adult onset NCL (also called type A Kufs disease) (Cannelii et al., Biochem Biophys Res Commun. 2009; 379(4):892-7, Arsov et al., Am J Hum Genet. 2011; 88(5):566-73). With vLINCL (referred to here as CLN6-Batten disease), age of onset is between 18 months and six years and death typically occurs by age 12-15. CLN6-Batten disease initially presents as impaired language and delayed motor/cognitive development in early childhood, with most patients being wheelchair-bound within four years of disease onset (Canafoglia et al., Neurology. 2015; 85(4):316-24). The disease progresses to include visual loss, severe motor deficits, recurrent seizures, dementia and other neurodegenerative symptoms.

CLN6 is a 311 amino acid protein with seven predicted transmembrane domains, and is predominately localized to the endoplasmic reticulum. As with other CLN proteins, its exact function remains unclear; however, it has been implicated in intracellular trafficking and lysosomal function. There are currently over 70 characterized disease-causing mutations in CLN6 (Warrier et al., Biochimica et Biophysica Acta. 2013; 1832(11):1827-30) with most of these mutations leading to either a complete loss of CLN6 protein or production of truncated CLN6 protein products that are thought to be highly unstable and/or non-functional. Several naturally-occurring animal models of CLN6-Batten disease have been described; these include sheep, canine and mouse models. The spontaneous mutation found in the Cln6^{nclf} mouse model (referred to herein as “Cln6^{nclf} mice”) recapitulates many of the pathological and behavioral aspects of the disease (Morgan et al., PLoS One. 2013; 8(11):e78694). The Cln6^{nclf} mice contain an insertion of an additional cytosine (c.307insC, frame shift after P102), resulting in a premature stop codon that is homologous to a mutation commonly found in CLN6-Batten disease patients (Gao et al., Am J Hum Genet. 2002; 70(2):324-35, Wheeler et al., Am J Hum Genet. 2002; 70(2):537-42).

Currently, there are no therapies that can reverse the symptoms of CLN6-Batten Disease. Thus, there is a need in the art for treatments for CLN6-Batten Disease.

### SUMMARY

Provided herein are methods and products for CLN6 gene therapy using recombinant AAV.

Provided herein are recombinant adeno-associated virus 9 (rAAV9) encoding a CLN6 polypeptide, comprising an rAAV9 genome comprising in 5′ to 3′ order: a hybrid chicken 3-actin (CB) promoter and a polynucleotide encoding the CLN6 polypeptide. In some cases, the rAAV9 genome comprises a self-complementary genome. Alternatively, the rAAV9 genome comprises a single-stranded genome.

Self-complementary recombinant adeno-associated virus 9 (scAAV9) are provided encoding the CLN6 polypeptide set out in SEQ ID NO: 1, in which the genome of the scAAV9 comprises in 5′ to 3′ order: a first AAV inverted terminal repeat, a hybrid chicken β-actin (CB) promoter comprising the sequence of SEQ ID NO: 3, a polynucleotide encoding the CLN6 polypeptide set out in SEQ ID NO: 2 and a second AAV inverted terminal repeat. The polynucleotide encoding the CLN6 polypeptide may be at least 90% identical to SEQ ID NO: 2.

Also provided are scAAV9 with a genome comprising in 5′ to 3′ order: a first AAV inverted terminal repeat, a CMV enhancer, a hybrid chicken 3-Actin promoter (cb), an SV40 intron, a polynucleotide encoding the CLN6 polypeptide of SEQ ID NO: 1 and a second AAV inverted terminal repeat; scAAV9 with a genome comprising in 5′ to 3′ order: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat; and scAAV9 with a genome comprising the gene cassette set out in the nucleic acid sequence of SEQ ID NO: 4.

Also provided are ssAAV9 with a genome comprising in 5′ to 3′ order: a first AAV inverted terminal repeat, a CMV enhancer, a hybrid chicken 3-Actin promoter (CB), an SV40 intron, a polynucleotide encoding the CLN6 polypeptide of SEQ ID NO: 1 and a second AAV inverted terminal repeat; ssAAV9 with a genome comprising in 5′ to 3′ order: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat; or ssAAV9 with a genome comprising the gene cassette set out in the nucleic acid sequence of SEQ ID NO: 4.

The nucleic acid sequence set out in SEQ ID NO: 4 is the gene cassette that is provided in FIG. 1A. Provided are rAAV9 comprising an scAAV9 genome or a ssAAV9 genome comprising a nucleic acid sequence that is at least 90% identical to the nucleic acid sequence of SEQ ID NO: 4, at least 95% identical to the nucleic acid sequence of SEQ ID NO: 4, or at least 98% identical to the nucleic acid sequence of SEQ ID NO: 4.

Further provided are nucleic acid molecules comprising a first AAV inverted terminal repeat, a CB promoter comprising the nucleic acid sequence of SEQ ID NO: 3, a nucleic acid sequence encoding the CLN6 polypeptide of SEQ ID NO: 1 and a second AAV inverted terminal repeat. In some embodiments, the polynucleotide encoding the CLN6 polypeptide may be at least 90% identical to the nucleic acid sequence of SEQ ID NO: 2.

Also provided are nucleic acid molecules comprising a first AAV inverted terminal repeat, a CB promoter comprising the nucleotide sequence of SEQ ID NO: 3, an SV40 intron, a nucleic acid sequence encoding the CLN6 polypeptide of SEQ ID NO: 1 and a second AAV inverted terminal repeat. In addition, provided are nucleic acid molecules comprising a first AAV inverted terminal repeat, a CB promoter comprising the nucleotide sequence of SEQ ID NO: 3, a nucleic acid encoding the CLN6 polypeptide of SEQ ID NO: 1, a BGH poly-A sequence and a second AAV inverted terminal repeat. In any of the polynucleotides provided, the CLN6 polypeptide can be encoded by a nucleic acid sequence at least 90% identical to the nucleic acid sequence of SEQ ID NO: 2.

Provided are rAAV with an scAAV genome or an ssAAV genome, wherein the genome comprises a nucleic acid sequence that is at least 90% identical to the nucleic acid sequence of SEQ ID NO: 4, or at least 95% identical to the nucleic acid sequence of SEQ ID NO: 4, or at least 98% identical to the nucleic acid sequence of SEQ ID NO: 4.

The provided rAAV can comprise any of the polynucleotides disclosed herein. In addition, viral particles comprising any of the disclosed nucleic acid s are provided. The rAAV with self-complementary or single-stranded genomes are also provided.

Also provided are recombinant adeno-associated virus 9 (rAAV9) viral particles encoding a CLN6 polypeptide, comprising an rAAV9 genome comprising in 5′ to 3′ order: a CMV enhancer comprising a nucleic acid sequence at least 90% identical to SEQ ID NO: 6, a CB promoter comprising a nucleic acid sequence at least 90% identical to SEQ ID NO: 3, and a polynucleotide encoding a CLN6 polypeptide at least 90% identical to the amino acid sequence of SEQ ID NO: 1. In some embodiments, the rAAV9 viral particles provided comprise a self-complementary genome. Alternatively, the rAAV9 viral particles provided comprise a single-stranded genome.

Further provided are rAAV9 viral particles, wherein the rAAV9 genome comprises in 5′ to 3′ order: a first AAV inverted terminal repeat, the CMV enhancer comprising a nucleic acid sequence at least 90% identical to SEQ ID NO: 6, the CB promoter comprising a nucleic acid sequence at least 90% identical to SEQ ID NO: 3, the polynucleotide encoding a CLN6 polypeptide at least 90% identical to the amino acid sequence of SEQ ID NO: 1, and a second AAV inverted terminal repeat. The rAAV9 particles provided comprise a polynucleotide encoding the CLN6 polypeptide comprising an amino acid sequence at least 90% identical to SEQ ID NO: 1. Any of the rAAV9 viral particles optionally further comprise an SV40 intron, and/or a BGH poly-A sequence.

In an additional embodiment, the rAAV9 viral particles comprise an AAV9 genome comprising a nucleic acid sequence at least 90% identical to the nucleic acid sequence of SEQ ID NO: 4, at least 95% identical to nucleic acid sequence of SEQ ID NO: 4, or at least 98% identical to the nucleic acid sequence of SEQ ID NO: 4.

In any of the rAAV, the ssAAV or the scAAV provided, the AAV inverted terminal repeats may be AAV2 inverted terminal repeats.

Also provided are nucleic acid molecules comprising an rAAV9 genome comprising in 5′ to 3′ order: a first AAV inverted terminal repeat, a CMV enhancer comprising a nucleic acid sequence at least 90% identical to SEQ ID NO: 6, a CB promoter comprising a nucleic acid sequence at least 90% identical to SEQ ID NO: 3, and a polynucleotide encoding a CLN6 polypeptide at least 90% identical to the amino acid sequence of SEQ ID NO: 1. The provided nucleic acid molecules comprise a self-complementary genome and/or a single stranded genome.

Further provided are nucleic acid molecules comprising a rAAV9 genome that comprises in 5′ to 3′ order: a first AAV inverted terminal repeat, the CMV enhancer comprising a nucleic acid sequence at least 90% identical to SEQ ID NO: 6, the CB promoter comprising a nucleic acid sequence at least 90% identical to SEQ ID NO: 3, the polynucleotide encoding a CLN6 polypeptide at least 90% identical to the amino acid sequence of SEQ ID NO: 1, and a second AAV inverted terminal repeat. The nucleic acid molecules provided can comprise a polynucleotide encoding the CLN6 polypeptide comprising an amino acid sequence at least 90% identical to amino acid sequence of SEQ ID NO: 1. In addition, the nucleic acid molecules can comprise an AAV9 genome comprising a nucleic acid sequence at least 90% identical to the nucleic acid sequence of SEQ ID NO: 4, at least 95% identical to nucleic acid sequence of SEQ ID NO: 4 or at least 98% identical to the nucleic acid sequence of SEQ ID NO: 4. Any of the nucleic acid molecules provided optionally further comprise an SV40 intron, and/or a BGH poly-A sequence.

Further provided are compositions comprising the scAAV9 described herein, nucleic acid molecules described herein or the rAAV viral particles described herein and at least one pharmaceutically acceptable excipient. In some instances, the pharmaceutically acceptable excipient comprises a non-ionic low osmolar compound, a buffer, a polymer, a salt, or a combination thereof. In some embodiments, the polymer is a copolymer. In some embodiments, the copolymer is a poloxamer. For example, the composition may at least comprise a pharmaceutically acceptable excipient comprising a non-ionic, low-osmolar compound. For example the pharmaceutically acceptable excipient may comprise about 20 to 40% non-ionic, low-osmolar compound or about 25% to about 35% non-ionic, low-osmolar compound. An exemplary composition comprises scAAV formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in 1×PBS comprising 0.001% Pluronic F68.

Still further provided are methods of treating CLN6-Batten Disease in a subject comprising administering to the subject a composition comprising a therapeutically effective amount of any of the rAAV9 disclosed herein, any of the scAAV9 disclosed herein, any of the ssAAV disclosed herein, any of the nucleic acid molecules described herein or any of the composition described herein.

The disclosure also provides use of a therapeutically effective amount of any of the rAAV9 disclosed herein, any of the scAAV9 disclosed herein, any of the ssAAV disclosed herein, any of the nucleic acid molecules described herein or any of the compositions described herein for the preparation of a medicament for treating CLN6-Batten Disease.

Also provided are compositions comprising a therapeutically effective amount of any of the rAAV9 disclosed herein, any of the scAAV9 disclosed herein, any of the ssAAV disclosed herein, any of the nucleic acid molecules described herein or any of the composition described herein for treating CLN6-Batten Disease.

In any of the methods, uses or compositions for treating CLN6-Batten Disease provided, the compositions, rAAV9, scAAV9, or ssAAV and/or nucleic acid molecules are administered via a route selected from the group consisting of intrathecal, intracerebroventricular, intraperenchymal, intravenous, and a combination thereof.

Exemplary doses of the scAAV9, ssAAV or rAAV9 administered by the intrathecal route are about 1×10¹¹ vg of the scAAV, ssAAV or rAAV9 viral particles to about 1×10¹⁵ vg of the scAAV or AAV9 viral particles, or about 1×10¹² vg of the scAAV, ssAAV or rAAV9 viral particles to about 1×10¹⁴ vg of the scAAV, ssAAV or AAV9 viral particles. For example, about 1×10¹³ vg of the scAAV, ssAAV or rAAV9 viral particles may be administered to a subject, or about 1.5×10¹³ the scAAV, ssAAV or rAAV9 viral particles may be administered to a subject, or about 6×10¹³ vg of the scAAV, ssAAV or rAAV9 viral particles may be administered to a subject.

The methods, uses or compositions for treating CLN6-Batten Disease disclosed herein result in a subject, in comparison to the subject before treatment or an untreated CLN6-Batten Disease patient, in one or more of: (a) reduced or slowed lysosomal accumulation of autofluorescent storage material, (b) reduced or slowed lysosomal accumulation of ATP Synthase Subunit C, (c) reduced or slowed glial activation (astrocytes and/or microglia) activation, (d) reduced or slowed astrocytosis, (e) reduced or slowed brain volume loss measured by MRI, (f) reduced or slowed onset of seizures, and (g) stabilization, reduced progression, or improvement in one or more of the scales used to evaluate progression and/or improvement in CLN6-Batten disease, e.g. Unified Batten Disease Rating System (UBDRS) assessment scales, the Hamburg Motor and Language Scale or the Mullen Scales of Early Learning (MSEL). The subject can be held in the Trendelenberg position after administering the rAAV9, the ssAAV9 viral particles, the scAAV or the nucleic acid molecules disclosed herein.

Still further provided are methods of treating CLN6 disease in a patient in need comprising delivering a composition comprising any one of the rAAV viral particles disclosed provided herein, any of the scAAV9 disclosed herein, any of the ssAAV9 disclosed herein, any of the nucleic acid molecules described herein or any of the composition described herein to a brain or spinal cord of a patient in need thereof.

In addition, the disclosure provides for use of any one of the rAAV viral particles disclosed provided herein, any of the scAAV9 disclosed herein, any of the ssAAV9 disclosed herein, any of the nucleic acid molecules described herein or any of the composition described herein for preparation of a medicament for use in delivering said ssAAV9, nucleic acid molecule, or composition to a brain or spinal cord of a patient in need thereof.

Also provided are compositions comprising any one of the rAAV viral particles disclosed provided herein, any of the scAAV9 disclosed herein, any of the ssAAV9 disclosed herein, any of the nucleic acid molecules described herein or any of the composition described herein for delivering said ssAAV9, nucleic acid molecule, or composition to a brain or spinal cord of a patient in need thereof.

In any of the methods, uses or compositions provided, the composition may be delivered by intrathecal, intracerebroventricular, intraparenchymal, or intravenous injection or a combination thereof. Any of the methods provided further comprise placing the patient in the Trendelenberg position after intrathecal injection of the composition, rAAV9, the ssAAV9 or the scAAV or the nucleic acid molecules disclosed herein.

In any of the methods, uses or compositions provided, the compositions or medicament may comprise a non-ionic, low-osmolar contrast agent. For example, the compositions may comprise a non-ionic, low-osmolar contrast agent is selected from the group consisting of iobitridol, iohexol, iomeprol, iopamidol, iopentol, iopromide, ioversol, ioxilan, and combinations thereof.

The compositions or medicaments administered may comprise a pharmaceutically acceptable excipient. For example, the pharmaceutically acceptable excipient may comprise about 20 to 40% non-ionic, low-osmolar compound or about 25% to about 35% non-ionic, low-osmolar compound. An exemplary composition comprises scAAV formulated in 20 mM Tris (pH8.0), 1 mM MgCl2, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

In any of the methods, uses or compositions provided, the composition or medicament may be delivered to the brain or spinal cord, the composition or medicament may be delivered to a brain stem, or may be delivered to the cerebellum, may be delivered to a visual cortex, or may be delivered to a motor cortex. Further, in any of the methods provided, the composition or medicament may be delivered to the brain or spinal cord, the composition may be delivered to a nerve cell, a glial cell, or both. For example, wherein the delivering to the brain or spinal cord comprises delivery to a cell of the nervous system such as a neuron, a lower motor neuron, a microglial cell, an oligodendrocyte, an astrocyte, a Schwann cell, or a combination thereof.

The methods, uses and compositions disclosed herein result in a subject, in comparison to the subject before treatment or in comparison to an untreated CLN6-Batten disease subject, in one or more of: (a) reduced or slowed lysosomal accumulation of autofluorescent storage material, (b) reduced or slowed lysosomal accumulation of ATP Synthase Subunit C, (c) reduced or slowed glial activation (astrocytes and/or microglia) activation, (d) reduced or slowed astrocytosis, (e) reduced or slowed brain volume loss measured by MRI, (f) reduced or slowed onset of seizures, and (g) stabilization, reduced progression, or improvement in one or more of the scales that are used to evaluate progression and/or improvement in CLN6-Batten disease, e.g., the Unified Batten Disease Rating System (UBDRS) assessment scales, the Hamburg Motor and Language Scale or the Mullen Scales of Early Learning (MSEL).

In any of the methods, compositions and uses described herein, the treatment, composition or medicament stabilizes or slows disease progression of CLN-6 Batten Disease. In particular, the disease progression is assessed with the UBDRS scales, the Hamburg Motor and Language Scale, the impact of treatment on quality of life using the Pediatric Quality of Life (PEDSQOL) scale, the Mullen Scales of Early Learning (MSEL), the potential for prolonged survival, or a combination thereof.

In any of the methods, uses or compositions described herein, the treatment, composition or medicament reduces or slows one or more symptoms of CLN-6 Batten Disease selected from: (a) loss of brain volume; (b) loss of cognitive function; and (c) language delay; as compared to an untreated CLN6-Batten Disease patient. In particular, the treatment stabilizes or slows disease progression of CLN-6 Batten Disease. For example, disease progression is assessed with the UBDRS scales, the Hamburg Motor and Language Scale, the impact of treatment on quality of life using the Pediatric Quality of Life (PEDSQOL) scale, the Mullen Scales of Early Learning (MSEL), the potential for prolonged survival, or a combination thereof.

In any of the method, uses or compositions described herein, the subject is aged 80 months or under, 75 months or under, 70 months or under, 65 months or under, 62 months or under, 60 months or under, 55 months or under, 50 months or under, or 40 months or under.

Given that there is no effective cure for CLN6-Batten disease, the Cln6^{nclf} mouse model was used to test the efficacy of introducing functional human CLN6 via adeno-associated virus (AAV)-mediated gene therapy. The pre-clinical results provided herein suggest that use of AAV-serotype 9 allows efficient expression of the human CLN6 protein throughout the CNS, where the most impacted cells are located. To evaluate safety of the treatment in a larger animal model, three four-year-old Cynomolgus Macaques were dosed with scAAV9.CB.CLN6 by intrathecal lumbar CSF injection and monitored for up to six months post-injection. No adverse effects or pathology were observed, while high levels of transgene expression were found throughout the brain and spinal cord of all animals. A single, postnatal intracerebroventricular (ICV) injection of scAAV9.CB.CLN6 into the CSF of mice induced persistent expression of the transgene in vivo in Cln6^{nclf} mice. Administration of scAAV9.CB.CLN6 reduced the classic hallmarks of the disease, including accumulation of autofluorescent storage material and ATP synthase subunit C, reactive gliosis, and loss of dendritic spines. Importantly, this gene therapy treatment leads to extensive functional benefits as it prevented many of the motor, memory and learning, and survival deficits of the Cln6^{nclf} mice. These results strongly underline the therapeutic potential of CSF-delivered scAAV9.CB.CLN6 for treatment of CLN6-Batten disease.

The headings herein are for the convenience of the reader and not intended to be limiting.

The use of ‘may’ and ‘can’ herein is to describe the various embodiments that are included within the claims, and not to indicate uncertainty about the scope of the claims.

### BRIEF DESCRIPTION OF THE DRAWINGS

FIGS. 1A-1C demonstrates neuronal targeting and expression of human CLN6 protein in vivo. FIG. 1A provides a schematic of the scAAV genome of scAAV.CB.CLN6. The graphs in FIG. 1B provide the CNL6 mRNA and human CLN6 (hCLN6) protein expression levels following transient transfection of HEK293 cells with scAAV.CB.CLN6 plasmid. The images in FIG. 1C provide immunohistochemical staining for GFP and hCLN6 protein after in utero electroporation of the scAAV.CB.CLN6 plasmid.

FIGS. 2A and 2B provide images showing widespread expression of the human CLN6 transcript in the CNS of Cln6^{nclf} mice injected with scAAV9.CB.CLN6. The images and graphs in FIG. 2A provide representative RT-PCR gels and quantitation by densitometry (normalized to GAPDH) at 6 months and 18 months post-injection. This analysis demonstrated increased gene expression following scAAV9.CB.CLN6 delivery (Cln6^{nclf}+scAAV9) compared to wild type mice (WT or WT+PBS) and PBS-injected Cln6^{nclf} mice (Cln6^{nclf}+PBS). The left panels of FIG. 2B provide images demonstrating widespread expression of the human CLN6 transcript in the CNS of Cln6^{nclf} mice injected with scAAV9.CB.CLN6 (Cln6^{nclf}+scAAV9) compared to wild type mice (WT+PBS) at 6 months and 18 months post-injection. The right panels of FIG. 2B provide images showing immunohistochemistry staining, which demonstrates protein expression in various brain regions of scAAV9.CB.CLN6-injected Cln6^{nclf} mice compared to wild type mice (WT+PBS) at 6 months and 18 months post-injection. Scale bar 50 m. Mean+/−SEM. N=3-9 mice/group. One-Way ANOVA, Bonferroni correction. *p<0.05, **p<0.01, ***p<0.001, ****p<0.0001.

FIGS. 3A-3C demonstrate the effect of a single scAAV9.CB.CLN6 injection in 2-month-old animals. The images and graphs in FIG. 3A provide representative RT-PCR gels and quantitation by densitometry (normalized to GAPDH) at 2 months post-injection. This analysis demonstrates increased gene expression following scAAV9.CB.CLN6 delivery (Cln6^{nclf}+scAAV9) compared to wild type mice (WT+PBS) and PBS-injected Cln6^{nclf} mice (Cln6^{nclf}+PBS). The top panels in FIG. 3B provide images demonstrating widespread expression of the human CLN6 transcript in the CNS of Cln6^{nclf} mice injected with scAAV9.CB.CLN6 (Cln6^{nclf}+scAAV9) compared to wild type mice (WT+PBS) at 2 months post-injection. The bottom panels of FIG. 3B provide images showing immunohistochemistry staining demonstrating protein expression in various brain regions of scAAV9.CB.CLN6-injected Cln6^{nclf} mice compared to wild type mice (WT+PBS) at 2 months post-injection. Scale bar 200 m. Mean+/−SEM. N=39. One-Way ANOVA, Bonferroni correction. *p<0.05, **p<0.01, ***p<0.001, ****p<0.0001. The images and graphs of FIG. 3C demonstrate that a single ICV injection of scAAV9.CB.CLN6 at P1 reduces accumulation of autofluorescent storage material (ASM; top panels) and ATP synthase subunit C (SubC; bottom panels) in the VPM/VPL and somatosensory cortex of Cln6^{nclf} mice compared to wild type mice (WT) and PBS-injected Cln6^{nclf} mice (Cln6^{nclf} PBS) 2 months after injection. Mean+/−SEM, N=3-10. (top panels); Mean+/−SEM, N=21-72, biological N=3-10 (bottom panels) One-Way ANOVA, Bonferroni correction. *p<0.05, **p<0.01, ***p<0.001, ****p<0.0001. Scale bar 200 m (top panels). Scale bar 50 m (bottom panels).

FIGS. 4A and 4B demonstrate widespread expression of CLN6 mRNA and hCLN6 protein throughout the brain in the following regions: A: motor cortex, B: somatosensory cortex; C: visual cortex; D: thalamus; E: pons; F: cerebellum; G: brainstem Images provided in FIG. 4A demonstrate hCLN6 transcript expression throughout the brain in scAAV9.CB.CLN6 treated Cln6^{nclf} mice at 2 months, 6 months and 18 months post-injection. Images provided in FIG. 4B demonstrate hCLN6 protein expression throughout the brain in scAAV9.CB.CLN6 treated Cln6^{nclf} mice at 2 months, 6 months and 18 months post-injection. Scale bar 50 μm.

FIG. 5 provides images and graphs demonstrating reduced accumulation of autofluorescent storage material (ASM) in the VPM/VPL and somatosensory cortex of scAAV9.CB.CLN6-treated Cln6^{nclf} mice (Cln6^{nclf} scAAV) compared to wild type mice (WT) and PBS-treated Cln6ⁿc mice (Cln6^{nclf} PBS) at 6 months and 18 months post-injection. Graphs show a number of ASM⁺ cells/2500 μm². Mean+/−SEM, N=3-10 based on time point. One-Way ANOVA, Bonferroni correction. *p<0.05, **p<0.01, ***p<0.001, ****p<0.0001. Scale bar 50 m.

FIG. 6 provides images and graphs demonstrating reduced accumulation of mitochondrial ATP synthase subunit C (SubUnitC) in the VPM/VPL and somatosensory cortex scAAV9.CB.CLN6-treated Cln6^{nclf} mice (Cln6^{nclf} scAAV) compared to wild type mice (WT) and PBS-treated Cln6^{nclf} mice (Cln6^{nclf} PBS) at 6 months and 18 months post-injection. Brown stain represents subunit C, while blue stain represents methyl green (nuclei). Graphs show total SubC⁺ area per image field. Mean+/−SEM, N=21-72, biological N=3-10. One-Way ANOVA, Bonferroni correction. *p<0.05, **p<0.01, ***p<0.001, ****p<0.0001. Scale bar 50 m.

FIG. 7 provides images and graphs demonstrating that scAAV9.CB.CLN6-injected Cln6^{nclf} mice (Cln6^{nclf} scAAV) exhibit less astrogliosis (GFAP reactivity) in the VPM/VPL and somatosensory cortex at 6 and 18M compared to wild type mice (WT) and PBS-injected Cln6^{nclf} mice (Cln6^{nclf} PBS). Graphs show total GFAP+ immunoreactivity. Mean+/−SEM, N=16-49 sections, biological N=3-10 mice/group. One-Way ANOVA, Bonferroni correction. *p<0.05, **p<0.01, ***p<0.001, ****p<0.0001. Scale bar 50 m. Inset scale bar 10 m.

FIG. 8 provides images and graphs demonstrating that scAAV9.CB.CLN6-injected Cln6^{nclf} mice (Cln6^{nclf} scAAV9) exhibit less microgliosis (CD68 reactivity) in the somatosensory cortex 6 months post-injection mice compared to wild type mice (WT) and PBS-injected Cln6^{nclf} mice (Cln6^{nclf} PBS), and in both the VPM/VPL and somatosensory cortex 18 months post-injection compared to wild type mice (WT) and PBS-injected Cln6^{nclf} mice (Cln6^{nclf} PBS). Graphs show total CD68+ immunoreactivity. Mean+/−SEM, N=16-49 sections, biological N=3-10 mice/group. One-Way ANOVA, Bonferroni correction. *p<0.05, **p<0.01, ***p<0.001, ****p<0.0001. Scale bar 50 m. Inset scale bar 10 m.

FIGS. 9A-9E provide graphs demonstrating that sustained expression of CLN6 rescues motor, memory, learning and survival deficits in Cln6^{nclf} mice. FIG. 9A demonstrates scAAV9.CB.CLN6-injected Cln6^{nclf} mice (Cln6^{nclf} scAAV) had reduced rotarod deficits from 8 to 24 months of age compared to wild type mice (WT) and PBS-injected Cln6^{nclf} mice (Cln6^{nclf} PBS). FIG. 9B demonstrates that scAAV9.CB.CLN6 injection corrects hind limb clasping, gait, and ledge lowering deficits in at 12 and 18M of age in scAAV9.CB.CLN6-injected Cln6^{nclf} mice (Cln6^{nclf} scAAV) compared to wild type mice (WT) and PBS-injected Cln6^{nclf} mice (Cln6^{nclf} PBS) FIG. 9C demonstrates that scAAV9.CB.CLN6 prevents memory and learning deficits in the Morris water maze from 9 to 12 months of age in scAAV9.CB.CLN6-injected Cln6^{nclf} mice (Cln6^{nclf} scAAV) compared to wild type mice (WT) and PBS-injected Cln6^{nclf} mice (Cln6^{nclf} PBS). FIG. 9D demonstrates that scAAV9.CB.CLN6 injection prevents early death of Cln6^{nclf} animals, while PBS-injected Cln6^{nclf} animals die by 15 months of age. FIG. 9E shows body weight development for males (left panel) and females (right panel) over the course of the study in scAAV9.CB.CLN6 treated mice (Cln6^{nclf} scAAV) compared to wild type animals (WT) and PBS injected Cln6^{nclf} mice (Cln6^{nclf} PBS). Mean+/−SEM, N=6-24 for rotarod, N=7-13 for clasping score, N=5-15 for water maze, N=10-15 for survival curve. N=3-13 for weight. One-Way ANOVA with Bonferroni correction or unpaired t-test used where appropriate. Log-rank (Mantel-Cox) test used for survival curve analysis *p<0.05, **p<0.01, ***p<0.001, ****p<0.0001

FIG. 10 provides additional behavior data in 12-24 month animals. The graph provided in FIG. 10A demonstrates that untreated Cln6^{nclf} animals have significantly slower swim speeds at 11 and 12 months of age in the Morris water maze test. The graph in FIG. 10B demonstrates that scAAV9.CB.CLN6 does not significantly improve memory and learning deficits of Cln6^{nclf} mice in the Morris water maze reversal task at 12, 18 and 24 months of age. Swim speeds are shown as a control. N=5-15 for water maze, unpaired t-test, Mean+/−SEM. *p<0.05, **p<0.01, ***p<0.001, ****p<0.0001

FIG. 11A-11C provides data demonstrating that scAAV9.CB.CLN6 is highly expressed and well-tolerated in non-human primates. FIG. 11A provides Western blots demonstrating high expression of the transgene in various brain and spinal cord regions of scAAV9.CB.CLN6-treated non-human primates. Blots are representative of 3 animals, with ‘+’ indicating an animal with scAAV9.CB.CLN6 treatment. The following brain regions were tested Cortex (Ctx), Corpus Callosum (C. Call), Periventricular White Matter (P.V.W.M.:), Hippocampus (Hipp), Cerebellum (Cere), Thalamus (Thal), Cervical Spinal Cord (Cervical), Thoracic Spinal Cord (Thoracic), Lumbar Spinal Cord (Lumbar). The graph in FIG. 11B provides quantification of fluorescent western blots in FIG. 1A. Mean+/−SEM, N=3. Unpaired student's t-test. *p<0.05, **p<0.01, ***p<0.001, ****p<0.0001. The graphs in FIG. 11C demonstrate that the delivery of scAAV9.CB.CLN6 did not alter platelet concentration or elevate liver enzymes in the majority of scAAV9.CB.CLN6-treated non-human primates. Red data points indicate scAAV9.CB.CLN6 treated animals; blue data points indicate PBS treated animals. The enzymes tested were as follows: Alanine Aminotransferase (ALT), Aspartate Aminotransferase (AST), Alkaline Phosphatase (Alk Phos) Gamma-Glutamyl Transferase (GGT).

FIGS. 12A-C provide analysis of diseases progression following injection of scAAV9.CB.CLN6 in two in-study sibling pairs as measured by the Hamburg Motor and Language Scale.

FIG. 13 provides the nucleic acid sequence of scAAV9.CB.CLN6 gene cassette (SEQ ID NO: 4). The AAV2 ITR nucleic acid sequence is in italics (5′ ITR is set out as SEQ ID NO: 9; 3′ ITR is set out as SEQ ID NO: 8), the CMV enhancer nucleic acid sequence (SEQ ID NO: 6) is underlined with a dotted line, the CB promoter nucleic acid sequence (SEQ ID NO: 3) is underlined with a single line, the SV40 intron nucleic acid sequence (SEQ ID NO: 11) is underlined with a double line, the nucleic acid sequence of the human CLN6 cDNA sequence (SEQ ID NO: 2) is in bold, the nucleic acid sequence of the BGH polyA terminator (SEQ ID NO: 10) is underlined with a dashed line.

FIG. 14 provides the nucleic acid sequence of full AAV.CB.CLN6 (SEQ ID NO: 8).

FIG. 15 provides efficacy data for 8 of the patients treated with scAAV9.CB.CLN6 as measured by the Hamburg Motor and Language Scale.

FIG. 16A-C provide comparison between treated and untreated siblings. One sibling was treated with scAAV9.CB.CLN6 and their progression as measured by the Hamburg Motor and Language Scale was compared to the natural history of their untreated sibling. This data is provided as Hamburg Score: Motor+Language over time.

FIG. 17 provides a Kaplan-Meier curve for the time until unreversed decrease from baseline of 2 or more points in the combined score for Hamburg Motor and Language function. This figure compares the data for the first 8 patients treated scAAV9.CB.CLN6 to data from an ongoing natural history study of CLN6 patients conducted by Nationwide Children's Hospital (n=14). Confidence bands are calculated using the survival probability estimates and their standard error.

FIG. 18 provides combined and individual Hamburg Motor and Language scores from patients treated with scAAV9.CB.CLN6 (n=8) showing that CLN6 gene therapy halts or substantially slows progression of disease with a positive impact on motor and language function in 7 out of 8 patients.

FIG. 19 provides natural history matched comparisons between patients treated with scAAV9.CB.CLN6 (n=8) compared to natural history patients matched for age and baseline Hamburg Motor and Language aggregate scores.

FIG. 20 provides natural history data for CLN6-Batten Disease patients (n=11). In the legend, the dotted line (----) indicates language decline and the solid gray line indicates motor decline. The blue line (top line) is the summation of both motor and language decline. The mean Hamburg Motor+Language score is plotted on the y-axis, and age in months is plotted on the x-axis. There is a fairly linear and almost sustained one-point decline per year from age two to seven.

FIG. 21A-B provide raw scores for 4 domains of the Mullen Early Learning Scale. Dotted horizontal lines indicate scores at screening. Higher scores indicate higher function.

### DETAILED DESCRIPTION

The present disclosure provides methods and products for treating CLN6-Batten Disease. The methods involve delivery of a CLN6 polynucleotide to a subject using rAAV as a gene delivery vector.

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

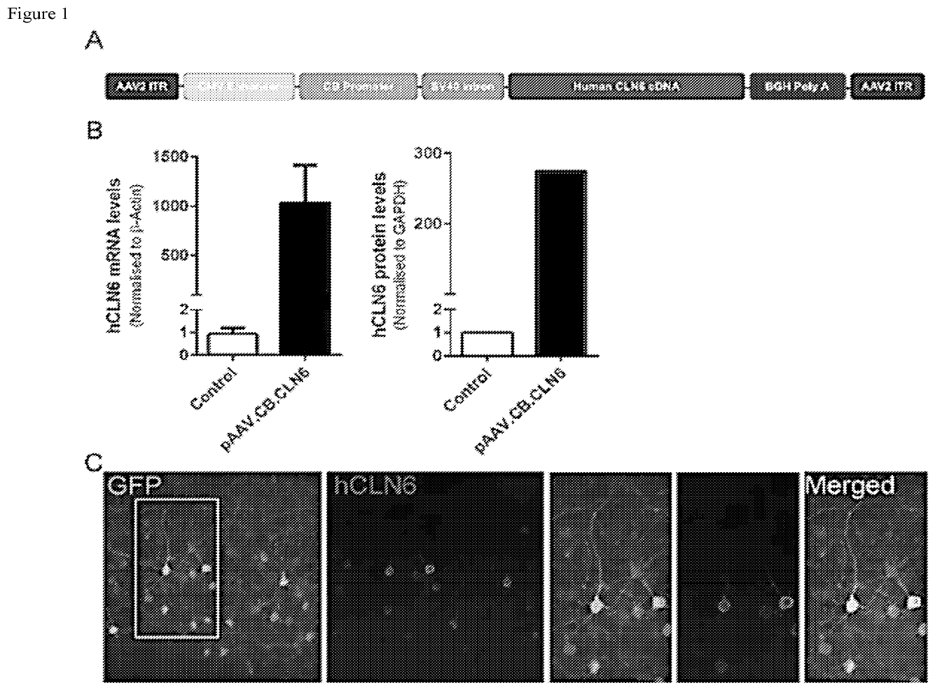

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest (e.g., a CLN6-encoding polynucleotide) flanked on the 5′ and 3′ ends by inverted terminal repeat (ITR). In some embodiments, the rAAV genome comprises a “gene cassette.” An exemplary gene cassette is set out in FIG. 1A and the nucleic acid sequence of SEQ ID NO: 4. The rAAV genome can be a self-complementary (sc) genome, which is referred to herein as “scAAV genome.” Alternatively, the rAAV genome can be a single-stranded (ss) genome, which is referred to herein as “ssAAV genome.”

The term “scAAV” refers to a rAAV virus or rAAV viral particle comprising a self-complementary genome. The term “ssAAV” refers to a rAAV virus or rAAV viral particle comprising a single-stranded genome.

rAAV genomes provided herein may comprise a polynucleotide encoding a CLN6 polypeptide. CLN6 polypeptides comprise the amino acid sequence set out in SEQ ID NO: 1, or a polypeptide with an amino acid sequence that is at least: 90%, 91%, 92%, 93%, 94%, 95%, 96%, 97%, 98%, or 99% identical to the amino acid sequence set forth in SEQ ID NO: 1, and which encodes a polypeptide with CLN6 activity (e.g., at least one of increasing clearance of lysosomal autofluorescent storage material, reducing lysosomal accumulation of ATP synthase subunit C, and reducing activation of astrocytes and microglia in a patient when treated as compared to, e.g., the patient prior to treatment).

rAAV genomes provided herein, in some cases, comprise a polynucleotide encoding a CLN6 polypeptide wherein the polynucleotide has the nucleotide sequence set out in SEQ ID NO: 2, or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 2 and encodes a polypeptide with CLN6 activity (e.g., at least one of increasing clearance of lysosomal autofluorescent storage material, reducing lysosomal accumulation of ATP synthase subunit C, and reducing activation of astrocytes and microglia in a patient when treated as compared to, e.g. the patient prior to treatment).

rAAV genomes provided herein, in some embodiments, comprise a polynucleotide sequence that encodes a polypeptide with CLN6 activity and that hybridizes under stringent conditions to the nucleic acid sequence of SEQ ID NO: 2, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

The rAAV genomes provided herein, in some embodiments, comprise one or more AAV ITRs flanking the polynucleotide encoding a CLN6 polypeptide. The CLN6 polynucleotide is operatively linked to transcriptional control elements (including, but not limited to, promoters, enhancers and/or polyadenylation signal sequences) that are functional in target cells to form a gene cassette. Examples of promoters are the chicken R actin promoter and the P546 promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate-early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Additionally provided herein are a CB promoter sequence set out in SEQ ID NO: 3, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 3 that are promoters with CB transcription promoting activity. Other examples of transcription control elements are tissue-specific control elements, for example, promoters that allow expression specifically within neurons or specifically within astrocytes. Examples include neuron-specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a CLN6 RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allows AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): an rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising an rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising an rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. An rAAV with an sc genome is referred to herein as a scAAV. The rAAV genome can be a single-stranded (ss) genome. An rAAV with a single-stranded genome is referred to herein as an ssAAV.

An exemplary rAAV provided herein is the scAAV named “scAAV9.CB.CLN6.” The scAAV9.CB.CLN6 scAAV contains a scAAV genome comprising a human CLN6 cDNA under the control of a hybrid chicken j-Actin (CB) promoter (SEQ ID NO: 3). The scAAV genome also comprises an SV40 Intron (upstream of human CLN6 cDNA) and Bovine Growth Hormone polyadenylation (BGH Poly A) terminator sequence (downstream of human CLN6 cDNA). The sequence of this scAAV9.CB.CLN6 gene cassette is set out in SEQ ID NO: 4. The scAAV genome is packaged in an AAV9 capsid and includes AAV2 ITRs (one ITR upstream of the CB promoter and the other ITR downstream of the BGH Poly A terminator sequence).

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions comprise a rAAV encoding a CLN6 polypeptide. Compositions may include two or more rAAV encoding different polypeptides of interest. In some embodiments, the rAAV is scAAV or ssAAV.

Compositions provided herein comprise rAAV and a pharmaceutically acceptable excipient or excipients. Acceptable excipients are non-toxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl2, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 1×10¹⁴, about 2×10¹⁴, about 3×10¹⁴, about 4×10¹⁴ about 5×10¹⁴, about 1×10¹⁵, to about 1×10¹⁶, or more total viral genomes. Dosages of about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.0×10¹⁴ vg are also contemplated. One dose exemplified herein is 6×10¹³ vg. Another dose exemplified herein is 1.5×10¹³.

Methods of transducing target cells (including, but not limited to, cell of the nervous system, nerve or glial cells) with rAAV are provided. The cells of the nervous system include lower motor neurons, microglial cells, oligodendrocytes, astrocytes, Schwann cells or combinations thereof.

The term “transduction” is used to refer to the administration/delivery of the CLN6 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a CLN6 polypeptide by an intrathecal, intracerebroventricular, intraparechymal, or intravenous route, or any combination thereof. Intrathecal delivery refers to delivery into the space under the arachnoid membrane of the brain or spinal cord. In some embodiments, intrathecal administration is via intracisternal administration.

Intrathecal administration is exemplified herein. These methods include transducing target cells (including, but not limited to, nerve and/or glial cells) with one or more rAAV described herein. In some embodiments, the rAAV viral particle comprising a polynucleotide encoding a CLN6 polypeptide is administered or delivered the brain and/or spinal cord of a patient. In some embodiments, the polynucleotide is delivered to brain. Areas of the brain contemplated for delivery include, but are not limited to, the motor cortex, visual cortex, cerebellum and the brain stem. In some embodiments, the polynucleotide is delivered to the spinal cord. In some embodiments, the polynucleotide is delivered to a lower motor neuron. The polynucleotide may be delivered to nerve and glial cells. The glial cell is a microglial cell, an oligodendrocyte or an astrocyte. In some embodiments, the polynucleotide is delivered to a Schwann cell.

In some embodiments of methods provided herein, the patient is held in the Trendelenberg position (head down position) after administration of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees).

The methods provided herein comprise the step of administering an effective dose, or effective multiple doses, of a composition comprising an rAAV provided herein to a subject (e.g., an animal including, but not limited to, a human patient) in need thereof. If the dose is administered prior to development of CLN6-Batten Disease, the administration is prophylactic. If the dose is administered after the development of CLN6-Batten Disease, the administration is therapeutic. An effective dose is a dose that alleviates (eliminates, stabilizes or reduces) at least one symptom associated with the disease, that slows or prevents progression of the disease, that diminishes the extent of disease, that results in remission (partial or total) of disease, and/or that prolongs survival. In comparison to the subject before treatment or in comparison to an untreated subject, methods provided herein result in stabilization, reduced progression, or improvement in one or more of the scales that are used to evaluate progression and/or improvement in CLN6 Batten-disease, e.g., the Unified Batten Disease Rating System (UBDRS), the Hamburg Motor and Language Scale or the Mullen Scales of Early Learning (MSEL). The UBDRS assessment scales (as described in Marshall et al., Neurology. 2005 65(2):275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival. In comparison to the subject before treatment or in comparison to an untreated subject, methods provided herein may result in one or more of the following: reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis, and showed a reduction or delay in brain volume loss measured by MRI.

Combination therapies are also provided. Combination, as used herein, includes either simultaneous treatment or sequential treatment. Combinations of methods described herein with standard medical treatments are specifically contemplated. Further, combinations of compositions (e.g., a combination of scAAV9.P546.CLN6 and a contrast agent disclosed herein) for use according to the invention—either simultaneous treatment or sequential treatment—are specifically contemplated.

While delivery to a subject in need thereof after birth is contemplated, intrauterine delivery to a fetus is also contemplated.

### EXAMPLES

While the following examples describe specific embodiments, it is understood that variations and modifications will occur to those skilled in the art. Accordingly, only such limitations as appear in the claims should be placed on the invention.

In the Examples, a self-complementary AAV (named scAAV9.CB.CLN6) carrying a CLN6 cDNA under the control of a hybrid chicken j-actin (CB) promoter was produced. IVC injection (6×10¹³ vg/animal) into the CSF of postnatal day 1 mice was sufficient to induce stable, robust expression CLN6 protein throughout the CNS for up to 18 months. Progression of CLN6-Batten disease is associated with the accumulation of ASM, aggregation of ATP synthase subunit C, decreased synaptic spine density, increased GFAP reactivity in astrocytes, and increased CD68 staining in microglia. Cln6^{nclf} mice display increases in ASM and ATP synthase subunit C and decreased dendritic spines at two months, and increased GFAP and CD68 reactivity by six months of age. Injection of scAAV9.CB.CLN6 in Cln6^{nclf} mice reduced accumulation of ASM and ATP synthase subunit C, increased dendritic spine density, and reduced levels of CD68+ microglial and GFAP+ astrocytic reactivity.

### Example 1

### Production of scAAV9.CB.CLN6

A human CLN6 cDNA clone was obtained from Origene, Rockville, Md. hCLN6 cDNA was further subcloned into an AAV9 genome under the hybrid chicken 3-Actin promoter (CB) and tested in vitro and in vivo. A self-complementary adeno-associated virus (scAAV) serotype 9 viral genome comprising the human CLN6 (hCLN6) gene under control of the chicken-3-actin (CB) hybrid promoter was generated. A schematic of the plasmid construct showing the CLN6 cDNA inserted between AAV2 ITRs is provided in FIG. 1A. The plasmid construct also includes the CP promoter, a simian virus 40 (SV40) chimeric intron and a Bovine Growth Hormone (BGH) polyadenylation signal (BGH PolyA).

scAAV9.CB.CLN6 was produced under cGMP conditions by transient triple-plasmid transfection procedures using a double-stranded AAV2-ITR-based CB-CLN6 vector, with a plasmid encoding Rep2Cap9 sequence as previously described (Gao et al., J. Virol., 78: 6381-6388 (2004)) along with an adenoviral helper plasmid pHelper (Stratagene, Santa Clara, Calif.) in HEK293 cells (36). The purity and titer of the vector were assessed by 4-12% sodium dodecyl sulfate-acrylamide gel electrophoresis and silver staining and qPCR analysis. After cloning, transgene expression was verified in vitro in HEK293 cells as well as in vivo via in utero ICV electroporation at embryonic day 15.5 (See FIG. 1B and FIG. 1C). This analysis confirmed neuronal targeting and expression of the human CLN6 protein in vivo.

### Example 2

### Analysis of Expression of CSF-Delivered scAAV9.CB.CLN6 in CLN6^{nclf} Mice

### Cell Targeting and Expression

To confirm the expression and biodistribution of virally-introduced human CLN6 in mice, scAAV9.CB.CLN6 was administered into CLN6ⁿC/J mice via a single intracerebroventricular (ICV) injection within 24 hours after birth and expression was monitored at various time points over a course of two months. Wild type and CLN6^{nclf} mice injected with an equal volume of PBS served as controls. The effective administered dose was 5×10¹⁰ vg/mouse using the NCH viral vector core titer. The scAAV9.CB.CLN6 was formulated in 1×PBS and 0.001% Pluronic F68 or formulated in 20 mM Tris (pH8.0), 1 mM MgCl2, 200 mM NaCl, 0.001% poloxamer 188.

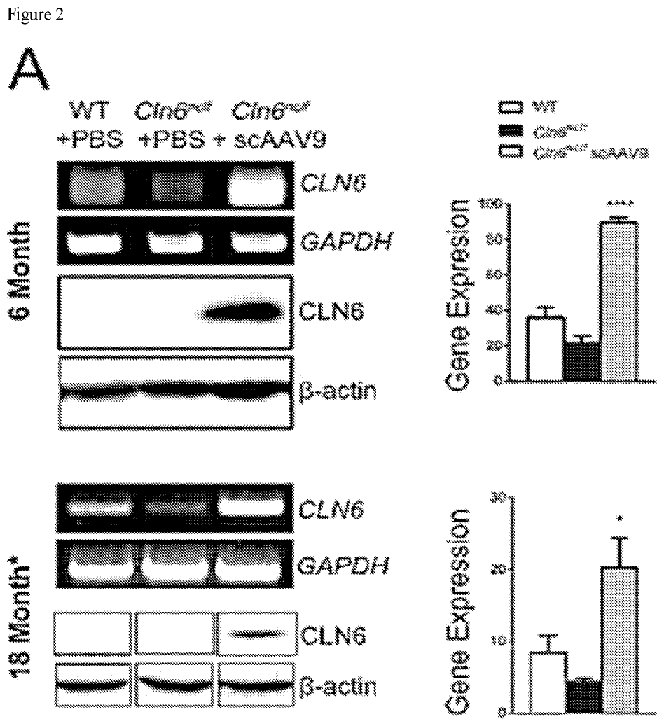

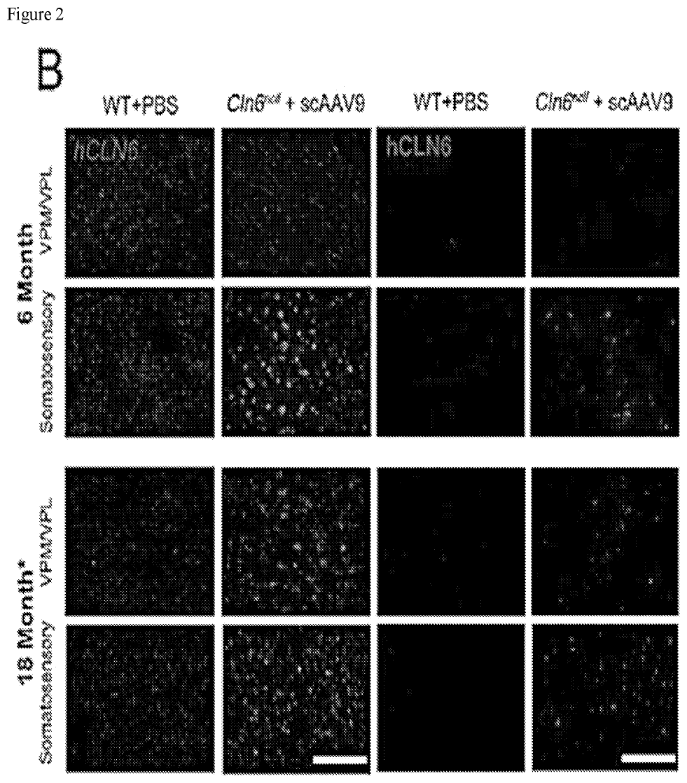

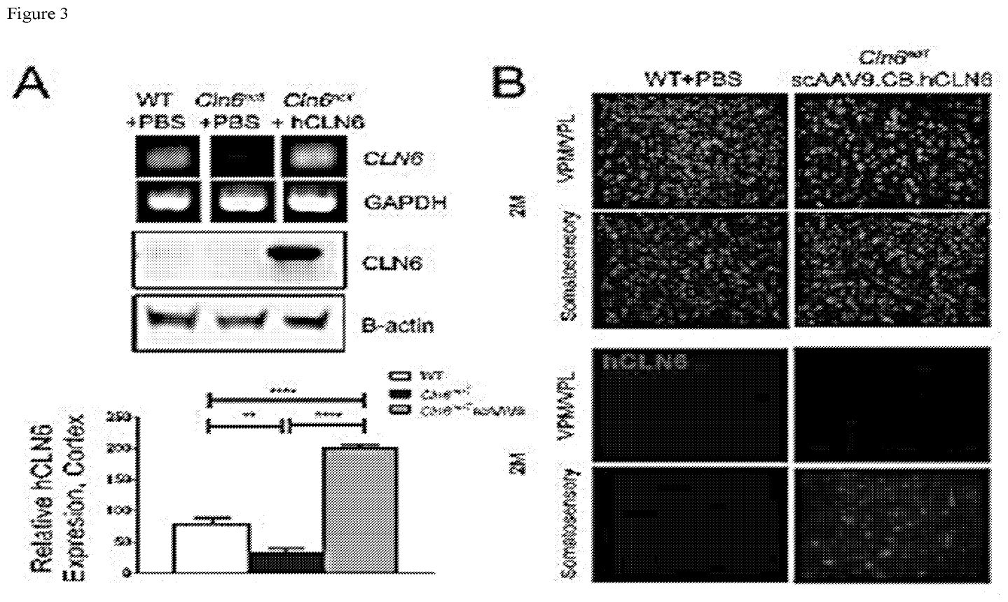

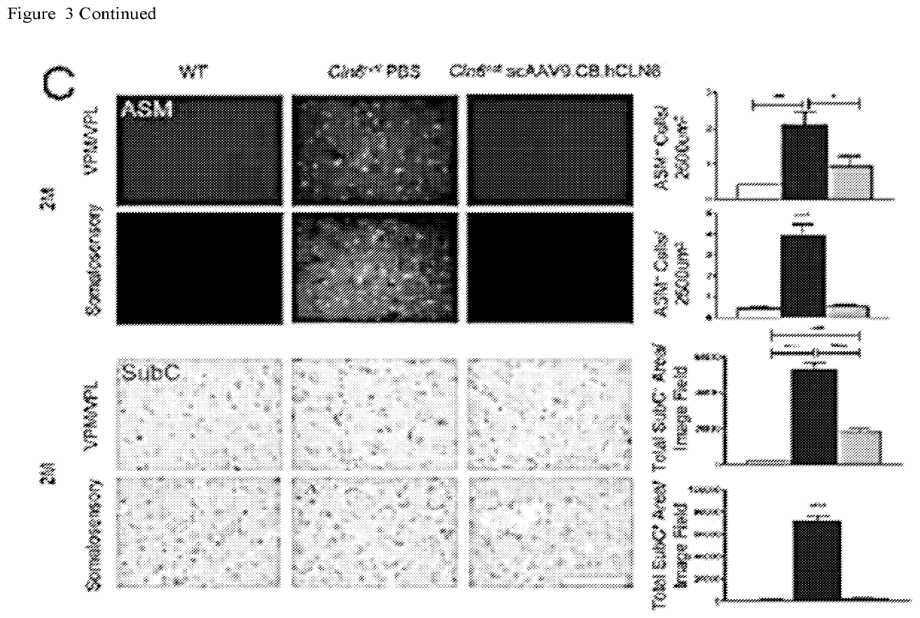

Examination of hCLN6 expression by RT-PCR at 2, 6, and 18 months post-injection demonstrated sustained, robust hCLN6 expression in the cortex of scAAV9.CB.CLN6-injected Cln6^{nclf} mice compared to PBS-injected controls (FIG. 2A, FIG. 3A). These results were similar to previously reported scAAV9-CB-GFP expression levels (See, e.g. Foust et al. Mol Ther. 2013; 21(12):2148-59, Foust et al., Nat Biotechnol. 2010; 28(3):271-4, Meyer et al., Mol Ther. 2015; 23(3):477-87) In FIG. 2A, the top gels and graphs are representative RT-PCR gels and densitometry (normalized to GAPDH). These data demonstrated increased gene expression following scAAV9.CB.CLN6 delivery compared to PBS-injected Cln6^{nclf} mice. The bottom gels and graphs show CLN6 protein expression as measured by western blotting. ICV delivery of the scAAV9.CB.CLN6 vector shows a marked increase in hCLN6 protein expression in the cerebral cortex of Cln6^{nclf} mice.

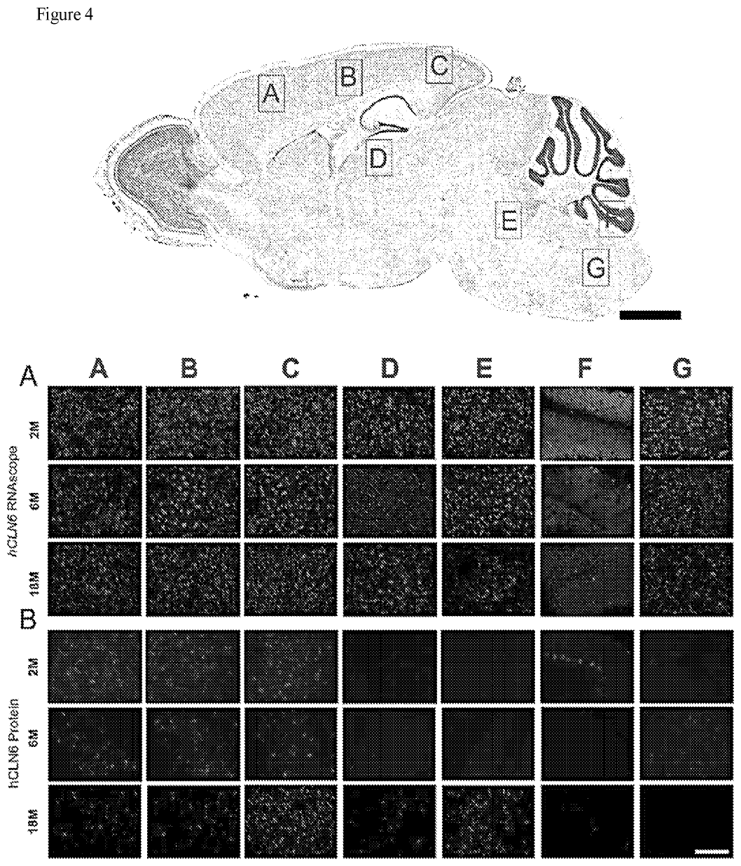

To examine the regional distribution of transgene expression, a modified in situ hybridization method called RNAScope® was used to visualize hCLN6 transcript. scAAV9.CB.CLN6-injected Cln6^{nclf} mice maintained a widespread transduction of hCLN6 throughout all regions of the brain at 2, 6 and 18 month, including the somatosensory cortex and VPM/VPL nuclei of the thalamus, two regions that have been shown to be affected earliest in the disease progression of the Cln6^{nclf} mice (FIG. 2B, left panels; FIG. 3B; top panels, FIG. 4A).

To examine expression of hCLN6 protein within the CNS, immunoblotting of cortical brain lysates harvested from scAAV9.CB.CLN6-injected Cln6^{nclf} and PBS-injected controls was performed using anti-hCLN6 antibodies. Identical to what was seen with RNA expression, robust hCLN6 protein expression was seen throughout the CNS at 2, 6, and 18 months of age (FIG. 2B, right panels, FIG. 3B, bottom panels). Furthermore, immunolabeling of brain tissue using anti-hCLN6 antibodies confirmed expression throughout the brain of scAAV9.CB.CLN6-treated Cln6^{nclf} mice (FIG. 4B). Together, these findings demonstrated that CSF delivery of scAAV9.CB.CLN6 via ICV injection was able to stably produce hCLN6 transcript and protein in disease-relevant regions of the CNS.

Pathology Improvements Following Delivery of scAAV9.CB.CLN6

### Accumulation of Autofluorescent Storage Material (ASM)

Accumulation of autofluorescent storage material (ASM) is the hallmark histological marker for Batten disease progression (Mole et al., Biochim Biophys Acta—Mol Basis Dis. 2015; 1852(10):2237-2241; Cotman et al., Clin Lipidol. 2012 February; 7(1):79-91; Seehafer et al., Neurobiol Aging. 2006; 27:576-588). Accumulation of ASM is a strong indicator for disease progression for many forms of Batten disease (Bosch et al., J Neurosci. 2016; 36(37):9669-9682; Morgan et al., PLoS One. 2013; 8(11):e78694). It is contemplated herein that reduction of ASM is used as indicator of successful treatment.

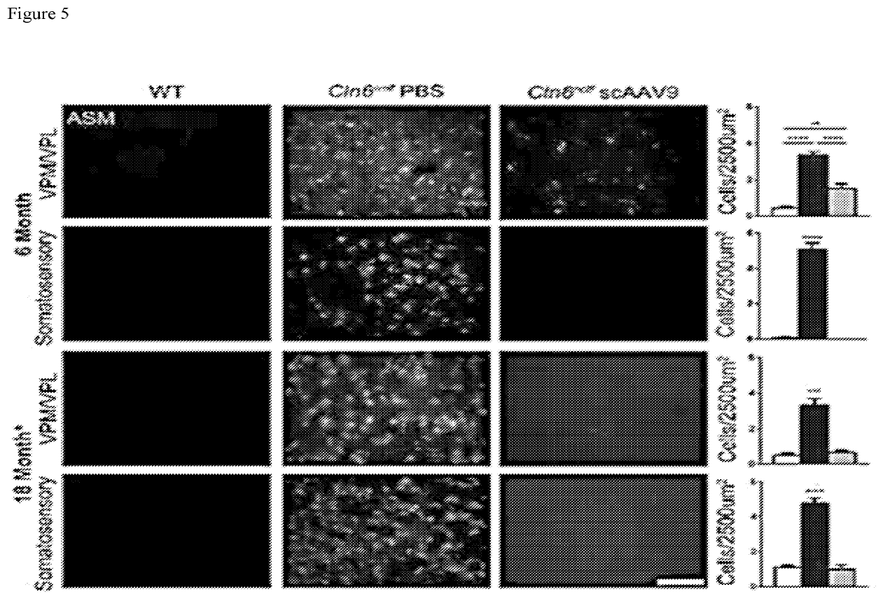

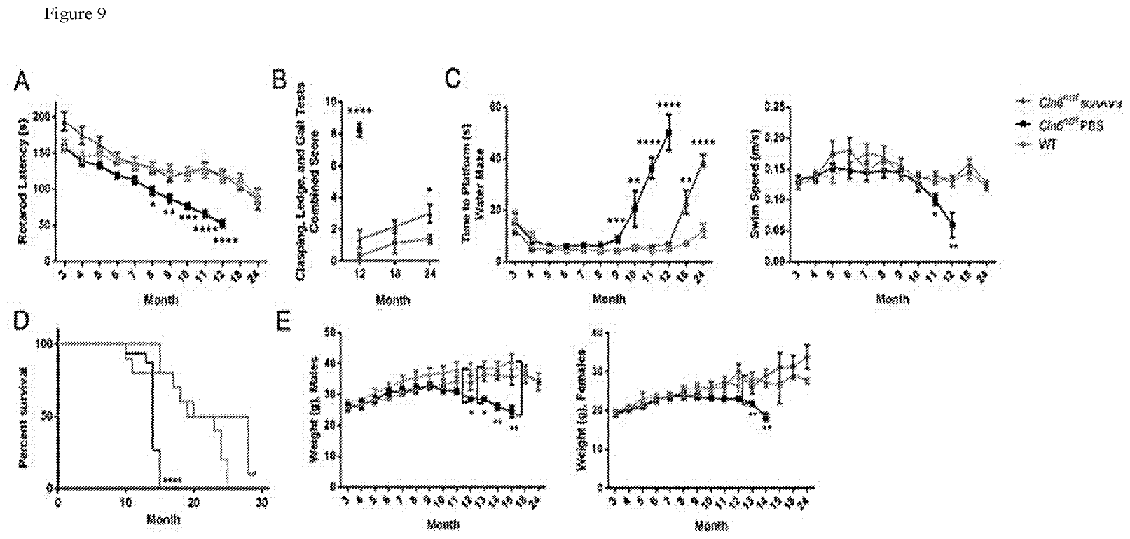

At 2, 6, and 18 months post-treatment, Cln6^{nclf} mice injected with scAAV9.CB.CLN6 had reduced accumulation of ASM within the VPM/VPL nuclei of the thalamus and somatosensory cortex of the brain compared to PBS-injected mice (FIG. 5, FIG. 3C). Because PBS treated Cln6nclf mice die by 15 months of age (FIG. 9D), moribund 12-14 month old PBS treated Cln6^{nclf} mice were used as a comparison to 18-month-old scAAV9.CB.CLN6 treated Cln6^{nclf} mice. Notably, the amount of ASM accumulation in these 18-month old scAAV9.CB.CLN6-injected Cln6^{nclf} mice was comparable to the age-matched untreated wild type mice. FIG. 9E demonstrates that male (left panel) and female (right panel) scAAV9.CB.CLN6 treated mice have similar body weights to wild type mice, age by age, while untreated Cln6^{nclf} mice decline over the course of the study. PBS injected Cln6^{nclf} mice (Cln6^{nclf} PBS) started losing weight around 10-11 months of age (males) and 13-14 months of age (females).

### Accumulation of Mitochondrial Protein ATP Synthase Subunit C

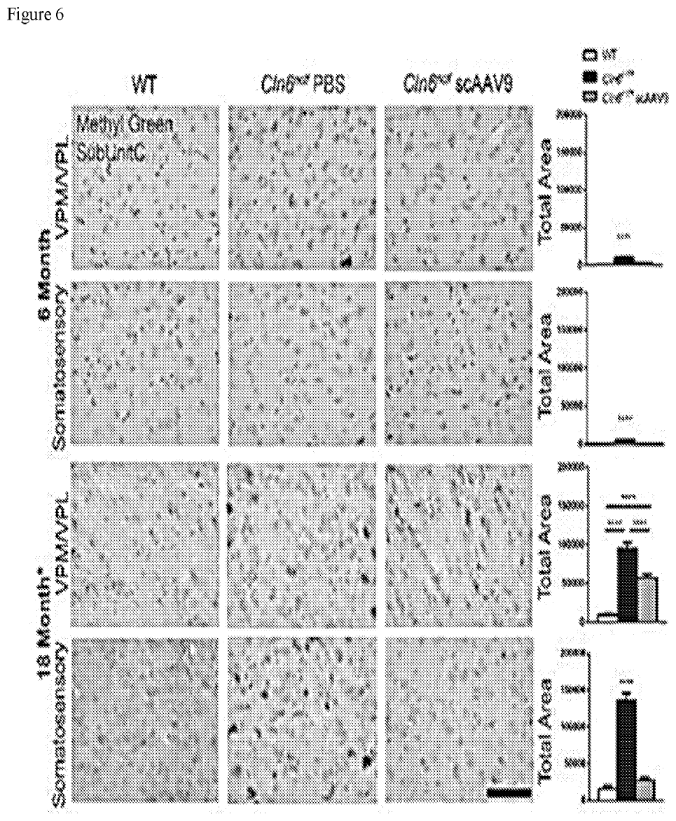

Accumulation of ATP synthase subunit C was analyzed in brain tissue from wild type, PBS-injected CLN6^{nclf} mice or scAAV9.CB.CLN6-injected Cln6^{nclf} mice. In healthy individuals, this protein is part of the respiratory chain in the mitochondrial membrane, but in patients suffering from Batten disease, the protein aberrantly accumulates in lysosomes (Palmer et al., Am J Med Genet. 1992; 42(4):561-567). In the Cln6^{nclf} mouse, compared to wild type animals, subunit C accumulation is apparent by 2 months of age in the ventral posteroiedial nucleus and ventral posterolateral nucleus of the thalamus (VPM/VPL region), a brain region often affected early on in NCL mouse models (Morgan et al., PLoS One. 2013; 8(11):e78694; Pontikis et al., Neurobiol Dis. 2005; 20(3):823-836). At 2, 6, and 18 months of age, Cln6^{nclf} mice treated with scAAV9.CB.CLN6 had significantly reduced levels of ATP synthase subunit C accumulation within the VPM/VPL and somatosensory cortex of the brain, compared to control Cln6^{nclf} mice injected with PBS (FIG. 6; FIG. 3C; bottom panels).

### Glial and Astrocyte Activation

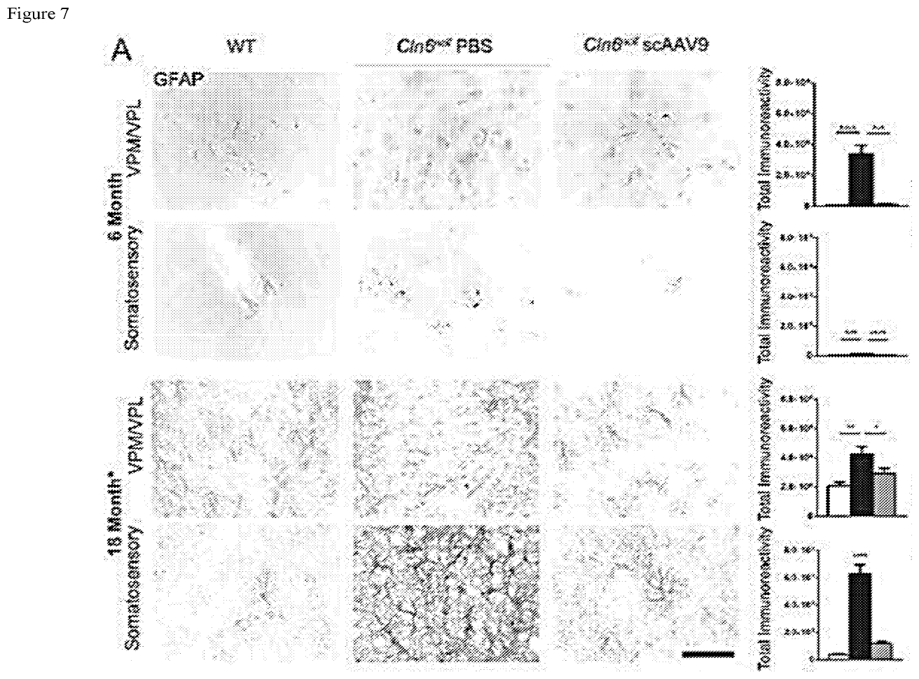

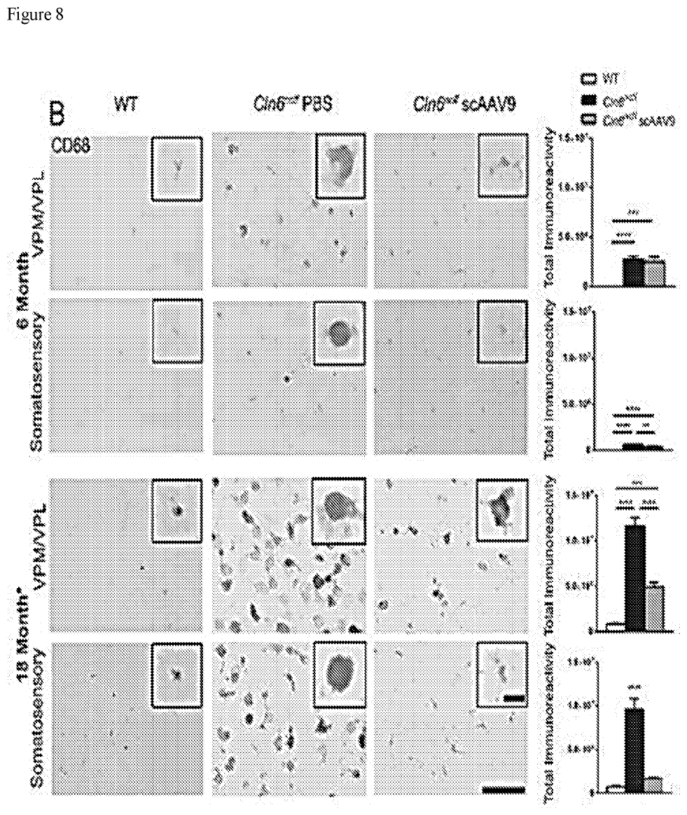

Besides aberrant accumulation of storage material and accumulation of ATP synthase sub C, other histological markers of disease progression in both human patients and animal models include activation of astrocytes and microglia (Cotman et al., Hum Mol Genet. 2002; 11(22):2709-2721; Morgan et al., PLoS One. 2013; 8(11):e78694; Pontikis et al., Neurobiol Dis. 2005; 20(3):823-836; Palmer et al., Am J Med Genet. 1992; 42(4):561-567). In particular, reactive microglia are primed to release pro-inflammatory mediators such as IL1-β26, which may be a key contributing cause of neuronal cell death at the later stages of CLN6-Batten disease. At 6 and 18 months of age, Cln6^{nclf} mice that were injected with scAAV9.CB.CLN6 had significantly reduced astrocyte activation (GFAP) and microgliosis (CD68) in the VPM/VPL and somatosensory cortex as compared to moribund PBS-treated Cln6^{nclf} mice (FIG. 7 and FIG. 8; respectively)

FIG. 7 demonstrates that activated astrocytes were identified in VPM/VPL thalamus and somatosensory cortex sections by staining for glial fibrillary acidic protein (GFAP) at 6 and 18 month time points. Graphs show total GFAP+ immunoreactivity.

Glial activation was also determined in VPM/VPL and somatosensory cortex sections using anti-CD68 staining as a marker for activated microglia. CD68 is a lysosomal protein that is upregulated in cells primed for pro-inflammatory functions such as phagocytosis (Seehafer et al., J Neuroimmunol. 2011; 230:169-172). FIG. 8 demonstrates that scAAV9.CB.CLN6 injection reduces microgliosis (CD68 reactivity) in the somatosensory cortex of 6M Cln6^{nclf} mice, and in both the VPM/VPL and somatosensory cortex of 18M Cln6^{nclf} mice. Graphs show total CD68+ immunoreactivity. The inlets in FIG. 8 showed morphology of microglia. It is worth noting that the untreated Cln6^{nclf} mice analyzed in these studies were moribund and many of the microglia were likely dying or dead, contributing to their unusual morphology. Together, these results indicated that a single injection delivering scAAV9.CB.CLN6 into the CSF at post-natal day 1 can reduce or delay many of the classic CLN6-Batten disease pathologies in the brains of Cln6^{nclf} mice.

Behavioral Improvements Following Delivery of scAAV9.CB.CLN6

In the efficacy study for scAAV9.CB.CLN6, starting at 2 months of age, and continuing at 2-month intervals, mice were subjected to a battery of behavioral testing paradigms including: accelerating rotarod assays, and pole climbing to test motor function and coordination, as well as Morris water maze to assess learning and memory. Animals were followed for 24 months post-injection and studies are ongoing.

### Rotarod Assay

Previous work demonstrated that the Cln6^{nclf} mouse model of CLN6-Batten disease recapitulates many of the motor, cognitive, and survival defects are seen in humans (Morgan et al., PLoS One. 2013; 8(11):e78694). In the efficacy study, using the rotarod as a classic measure of motor coordination, PBS-injected Cln6^{nclf} mice began to show a decline in rotarod performance at 8 months of age compared to wild type. However, injection of Cln6^{nclf} mice with scAAV9.CB.CLN6 prevented this decline, an effect that lasted for the duration of the entire study period (24 months) (FIG. 9A). To further study the effects of motor coordination in detail, animals were subjected to various motor tasks (hind limb clasping, ability to lower oneself from a ledge, and gait assessment) at 12, 18, and 24 months of age and assessed using a scoring matrix, with the highest score being the worst prognosis (Guyenet et al., Journal of visualized experiments: JoVE. 201039). Compared to PBS-treated Cln6^{nclf} mice, mice treated with scAAV9.CB.CLN6 showed significantly lower combined scores at all time points, with a slight increase in their score only at 24 months of age (FIG. 9B).

### Morris Water Maze Test

In the Morris Water Maze test, animals were placed in a water-filled pool containing a hidden platform. After training, the time it took the animals to find the hidden platform using environmental cues for orientation was measured as a sign of learning and memory capabilities.

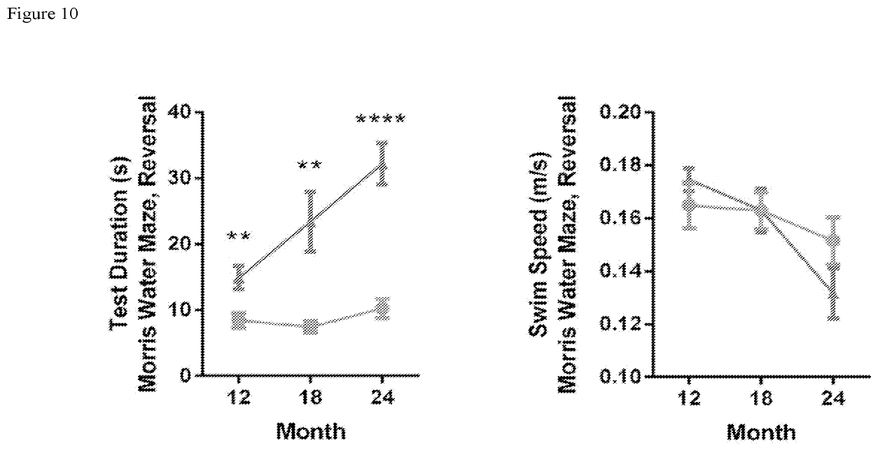

PBS-treated Cln6^{nclf} mice performed poorly at the task starting at nine months of age, indicated by their reduced ability to find the hidden platform (FIG. 9C). Since the swim speeds of PBS-treated Cln6^{nclf} mice were significantly reduced at 11 and 12 months of age, we could not draw any conclusions on their memory and learning abilities at these later time points (FIG. 10A). Treatment of Cln6^{nclf} mice with scAAV9.CB.CLN6 corrected this memory and learning deficit up to 12 months post-injection (FIG. 9C). When comparing wild type mice to scAAV9.CB.CLN6 treated animals only at later time points in the Morris water maze test, we found that even the treated mice needed more time to find the platform at 18 and 24 months, while the swim speed was the same between all test groups (FIG. 9C, FIG. 10). To assess memory and learning at later time points, mice were subjected to a water maze reversal test at 12, 18, and 24 months of age where the platform was moved to a novel location. Cln6^{nclf} mice treated with scAAV9.CB.CLN6 took significantly longer to find the new platform location compared to wild type mice in this test as well (FIG. 10). Taken together, these results indicate that a single treatment of scAAV9.CB.CLN6 prevented many of the motor declines seen in these animals, but does not fully ward off memory and learning deficits when the mice were tested at later time points.

Improvement in Survival Following Delivery of scAAV9.CB.CLN6

Cln6^{nclf} mice are known to have reduced survival compared to their wild type counterparts (Guyenet et al., Journal of visualized experiments: JoVE. 201039). Survival of scAAV9.CB.CLN6 and PBS-injected Cln6^{nclf} mice was compared with PBS-injected wild type mice. A single ICV injection of scAAV9.CB.CLN6 into the CSF of Cln6^{nclf} mice significantly increased their survival compared to PBS-injected Cln6^{nclf} mice (FIG. 9D). While the median survival of PBS treated mice was 14 months, scAAV9.CB.CLN6-treated Cln6^{nclf} mice had a median survival of 21.5 months. This 65% increase in survival rate was highly significant. Moreover, the survival curve of scAAV9.CB.CLN6-treated Cln6^{nclf} mice was not significantly different from wild type animals.

Further, as a measure of overall health, body weight was recorded monthly. The improvement in health and survival was also underlined by the ability of scAAV9.CB.CLN6 treated mice to maintain their body weight, as no difference was observed compared to wild type animals, while PBS treated Cln6^{nclf} started losing weight around months 10-12 (FIG. 9E).

A safety study with 172 wild type mice treated with PBS and 223 wild type mice treated with 5×10¹⁰ vg/animal was carried out. This study demonstrated that scAAV9.CB.CLN6 was well tolerated up to 24 weeks with no adverse effects attributable to the virus (data not shown). Taken together, this is the longest survival extension in the Cln6^{nclf} mouse model to date, and indicate the utility of a single treatment of scAAV9.CB.CLN6 to restore both cellular and functional deficits of CLN6-Batten disease.

### Example 3

### Safety Study of scAAV9.CB.CLN6 in Non-Human Primates

To test safety of this treatment in a large animal model more relevant to human patients, 3 four-year old male Cynomolgus Macaques were administered scAAV9.CB.CLN6 formulated in 1×PBS and 0.001% Pluronic F68.

The animals were sacrificed at 1, 3 or 6 months post-injection. Each individual received a single lumbar intrathecal injection, delivering the viral vector directly into the CSF at a dose of 6×10¹³ viral particles per animal. After the injection, the animals were held in a Trendelenburg position for 15 minutes with head facing downwards in a 45-degree angle to facilitate targeting of the brain and upper spinal cord areas.

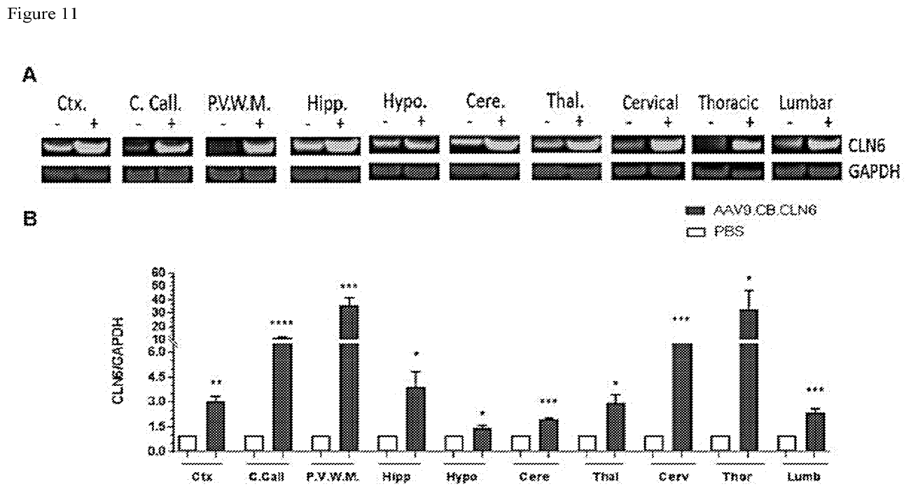

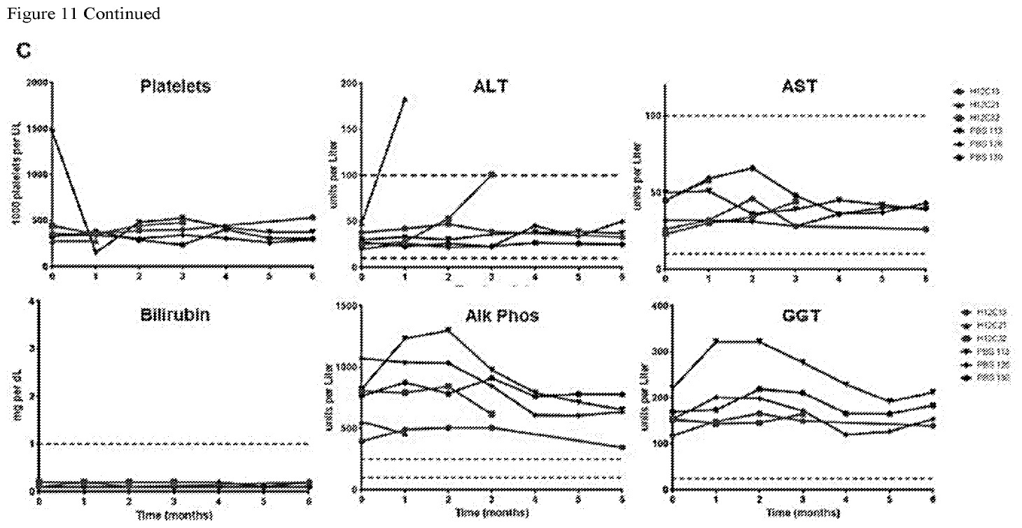

All subjects recovered well from the injection and did not show any abnormal behavior. Hematology and Serum Chemistry was performed at up to 5-time points during the study (baseline, 1, 2, 3 and 6 months) and did not reveal major abnormalities. In particular, no evidence of elevation in aspartate aminotransferase (AST) or alkaline phosphate enzyme levels were found, while alanine aminotransferase (ALT) was slightly increased in one animal at 1 month post injection (below 200 Units per Liter) (FIG. 11C).

No changes were found in total protein levels, creatinine, triglycerides, glucose or ions such as phosphorus, calcium, magnesium or sodium levels. Extensive histopathology as well as transgene expression analysis was performed for each animal at the time they were sacrificed. No abnormalities were found in any tissue analyzed including various brain and spinal cord regions, heart, lung, liver, spleen, kidney, small intestine, skeletal muscles (diaphragm, triceps, TA, gastrocnemius), gonads except one animal that displayed a bladder infection at time of necropsy.

The single lumbar intrathecal injection delivering scAAV9.CB.CLN6 into the cerebral spinal fluid induced high expression of the transgene throughout the brain and spinal cord of non-human primates, as shown by fluorescent western blot. The blots in FIG. 11A show CLN6 expression in the cortex, the corpus callosum, periventricular white matter, hippocampus, cerebellum, thalamus, cervical spinal cord, thoracic spinal cord, lumbar spinal cord high expression of the transgene was found throughout the brain and spinal cord in all three animals (FIG. 11A-B). Together, these data indicated that the treatment with scAAV9.CB.CLN6 was well tolerated and safe in all three individuals tested.

### Example 4

### Clinical Trial of scAAV9.CB.CLN6 Gene Therapy

The scAAV9.CB.CLN6 is delivered intrathecally to human patients with CLN6-Batten Disease.

The scAAV for the clinical trial was produced by the Nationwide Children's Hospital Clinical Manufacturing Facility utilizing a triple-transfection method of HEK293 cells, under GMP conditions as described in Example 1.

Patients selected for participation were one year or older in age with a diagnosis of CLN6 disease as determined by genotype. The first cohort (n=12) received a one-time gene transfer a dose of 1.5×10¹³ vg total scAAV per patient. The scAAV9.CB.CLN6 was formulated 20 mM Tris (pH8.0), 1 mM MgCl2, 200 mM NaCl, 0.001%. poloxamer 188 and about 20% to about 40% non-ionic, low-osmolar compound and was delivered one-time through an intrathecal catheter inserted by a lumbar puncture into the interspinous into the subarachnoid space of the lumbar thecal sac. Safety was assessed on clinical grounds, and by examination of safety labels. There was a minimum of four weeks between enrollments of each subject to allow for a review of Day 30 post-gene transfer safety data.

Preliminary data provided herein reports on ten patients that were treated and the average follow-up duration was 12 months (ranging 1-24 months post-treatment). The preliminary data demonstrated that administration of scAAV9.CB.CLN6 was generally well-tolerated. The majority of adverse events were mild and unrelated to treatment. Any T-cell response and antibody elevation observed were not associated with clinical manifestations and no changes in treatment were required.

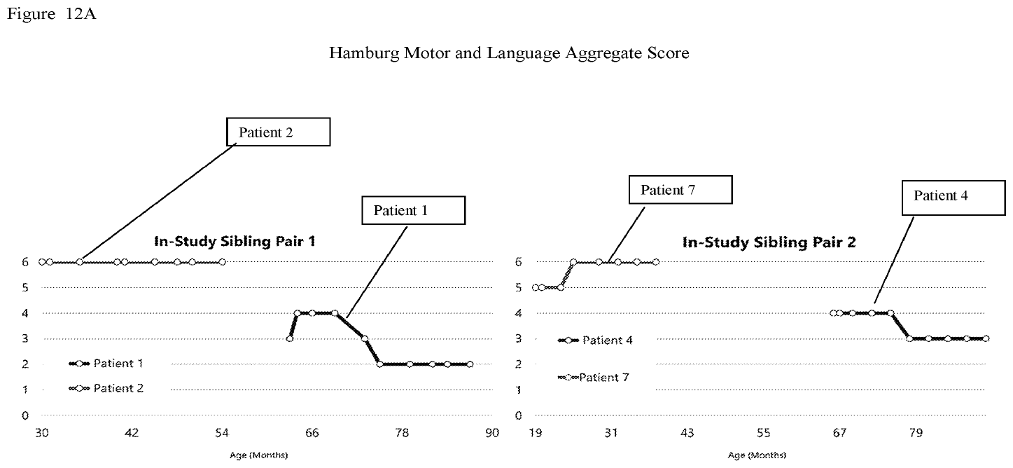

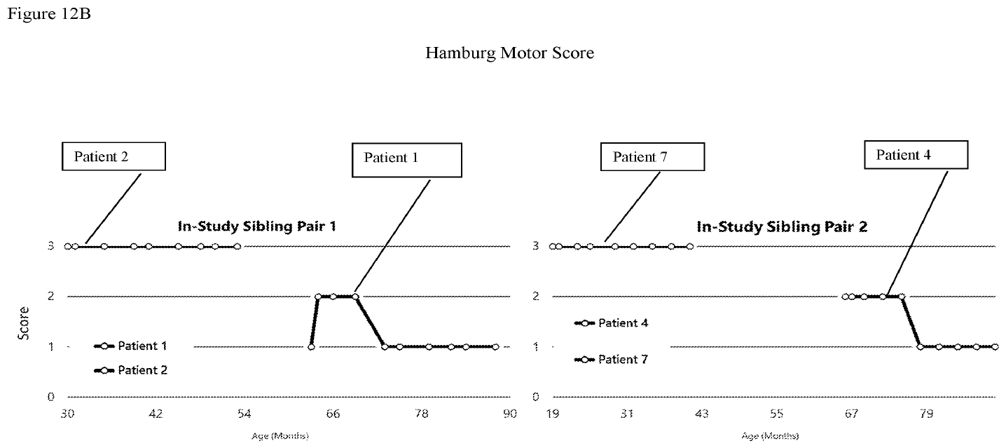

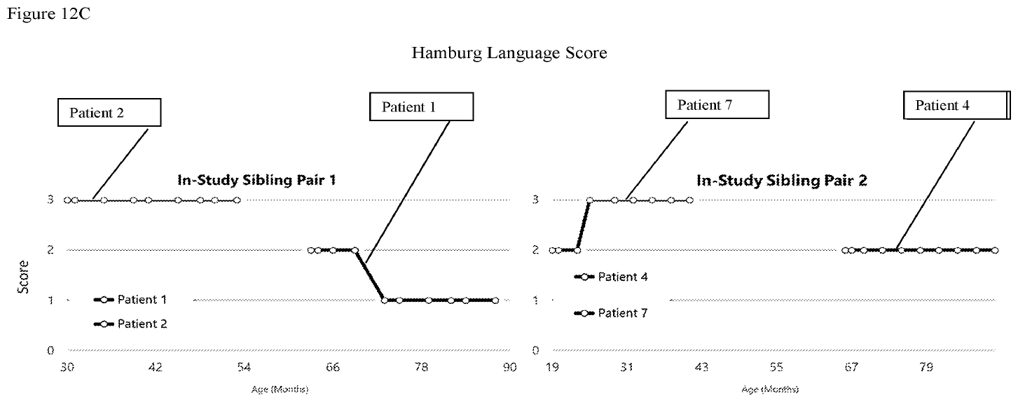

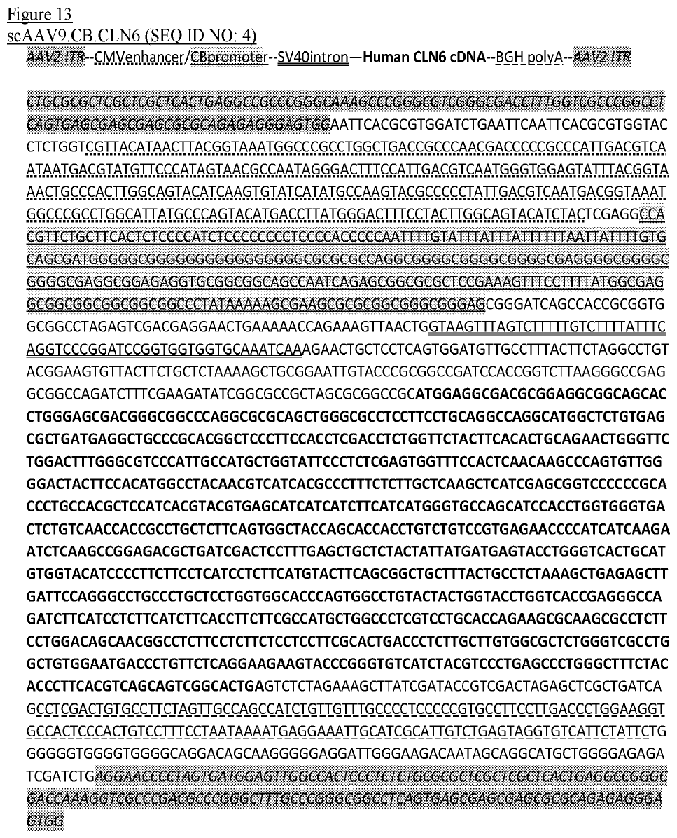

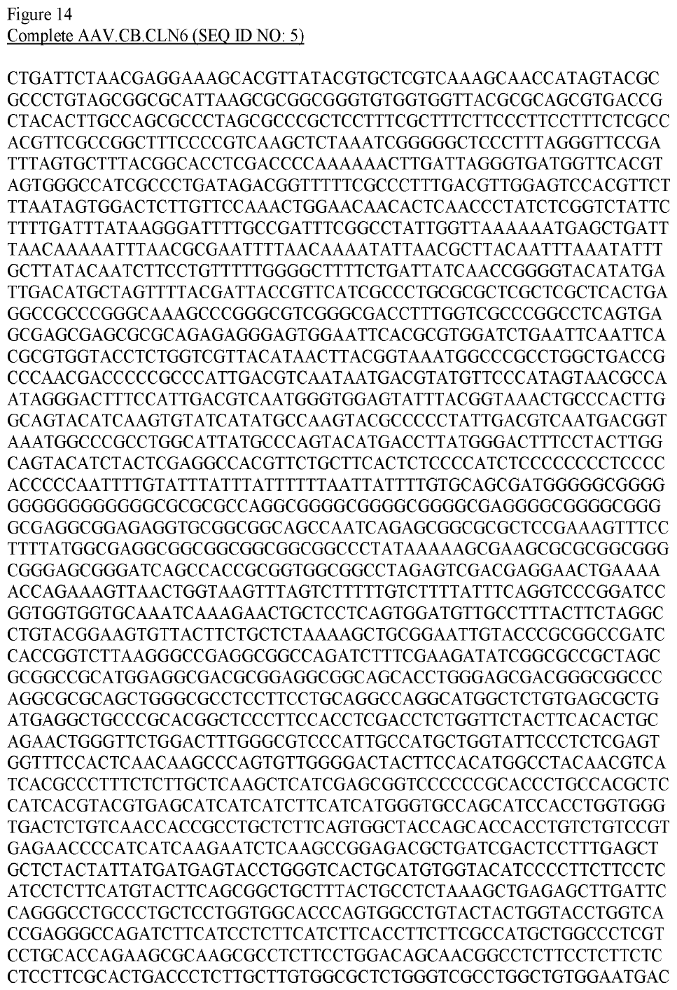

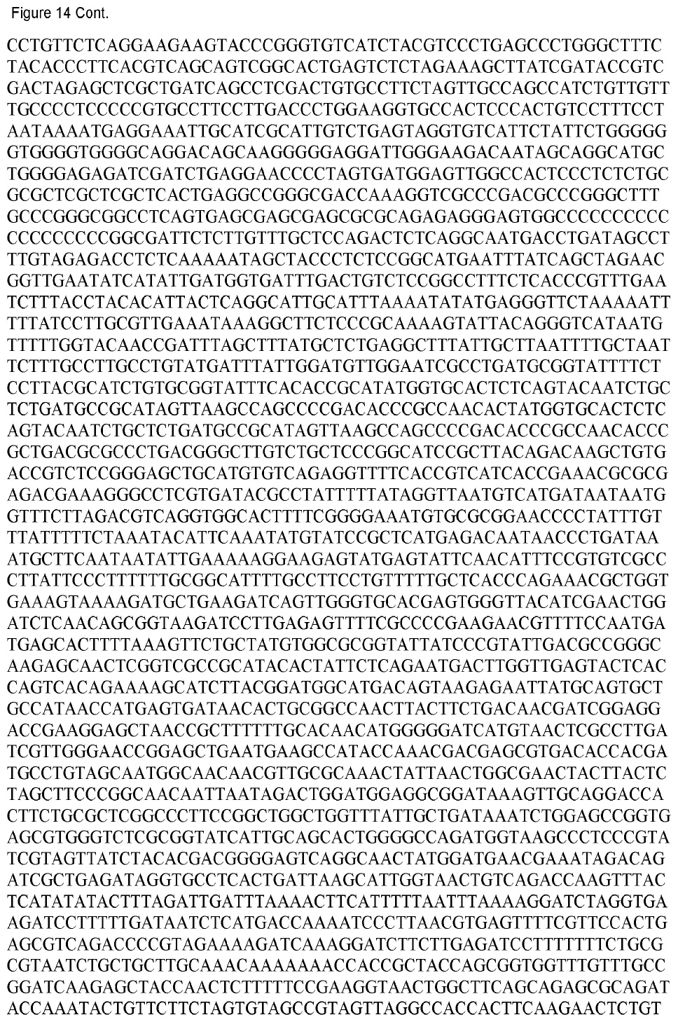

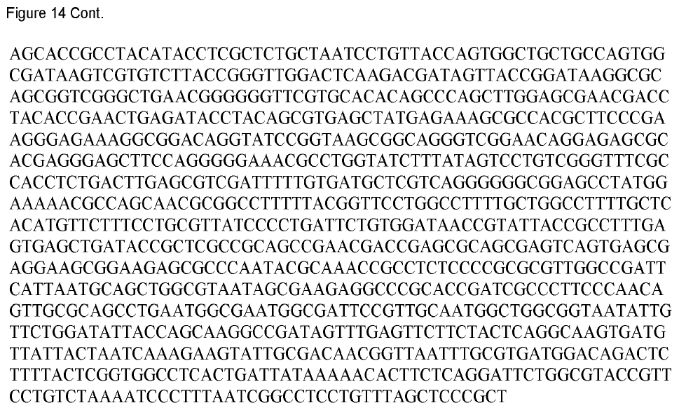

FIG. 12 provides preliminary data reporting disease progression as measured by the Hamburg Motor and Language Scale post-injection in in-study two in-study sibling pairs. These sibling pairs have the same gCLN6 mutation genotype.

### Twenty-Four Month Phase Efficacy Study

Provided herein are the data for eight of the treated patients for the ongoing clinical study. The 8 patients described herein were administered and exposed to scAAV9.CB.CLN6 for at least 17 months. The baseline information for these 8 patients is provided below.

| | | | | Hamburg | Time Between |
|---|---|---|---|---|---|
| | | Age at | | Motor + | Baesline and |
| Pa- | | Enrollment | Exposure | Language at | Last Measure |
| tient | Gender | (months)* | Duration | Baseline | (Months) |
| 1 | F | 63 | 39 | 3 | 25 |
| 2 | F | 30 | 38 | 6 | 23 |
| 3 | M | 36 | 36 | 5 | 24 |
| 4 | M | 66 | 28 | 4 | 24 |
| 5 | F | 79 | 27 | 3 | 24 |
| 6 | M | 56 | 26 | 5 | 24 |
| 7 | M | 19 | 20 | 5 | 19 |
| 8 | M | 61 | 17 | 4 | 16 |

Data from the ongoing 24-month clinical study indicated that a single intrathecal administration of scAAV9.CB.CLN6 was generally well tolerated. There were 137 adverse events reported. The majority of adverse events (AEs) were mild and unrelated to treatment. There were 9 grade 3 (severe) adverse events reported in 4 patients (denoted as SAEs). Three of 9 SAEs were considered to be possibly related to treatment. The related events included vomiting (2), epigastric pain (1), and fever (1) and all four patients recovered. There were no grade 4 (life-threatening) or grade 5 (death) adverse events reported. No pattern of adverse events related to anti-AAV9 capsid or anti-CLN6 immunogenicity.

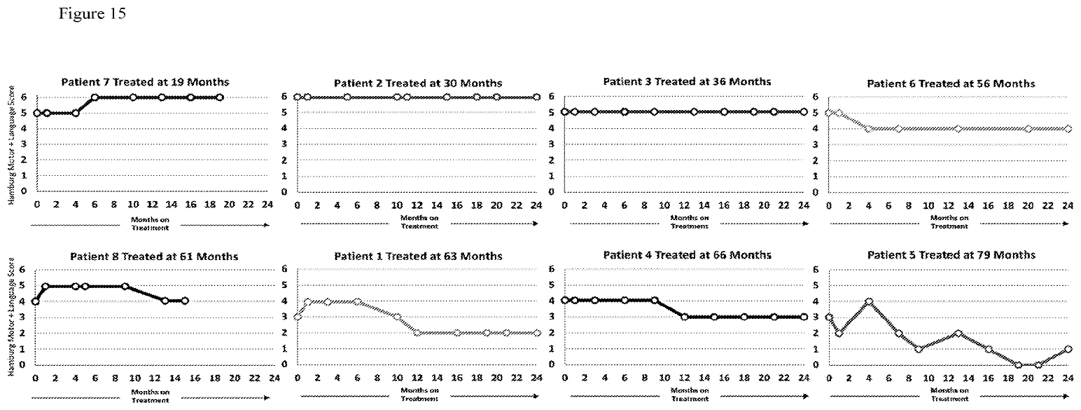

FIG. 15 provides efficacy data which shows a positive impact on motor and language function. In 7 of 8 of the patients treated with scAAV9.CB.CLN6, the Hamburg score was maintained or had an initial change (+1 to −1 points) followed by stabilization. The oldest patient in this study (treated at 79 months of age) had a two-point decline. Natural history data suggested a 2-3 point decline in Hamburg Motor and Language over 24 months post symptom onset.

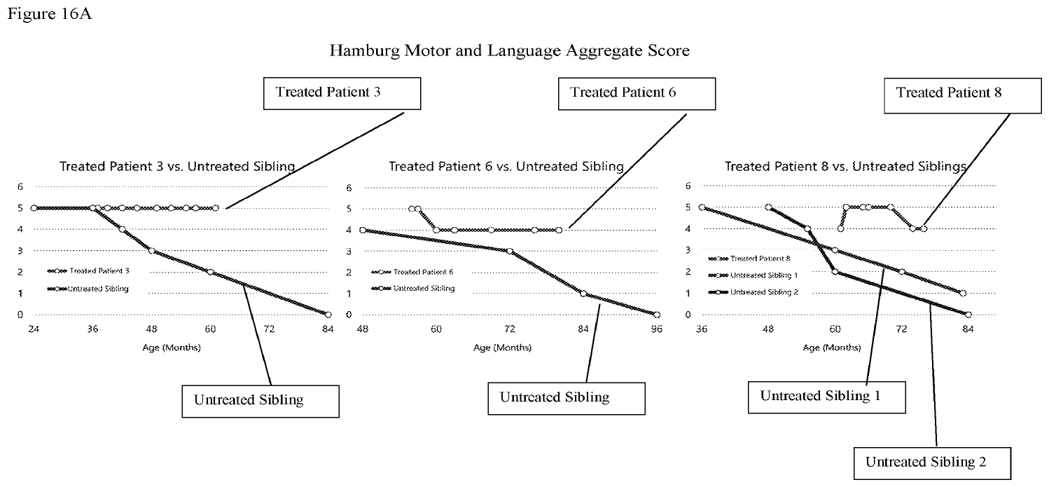

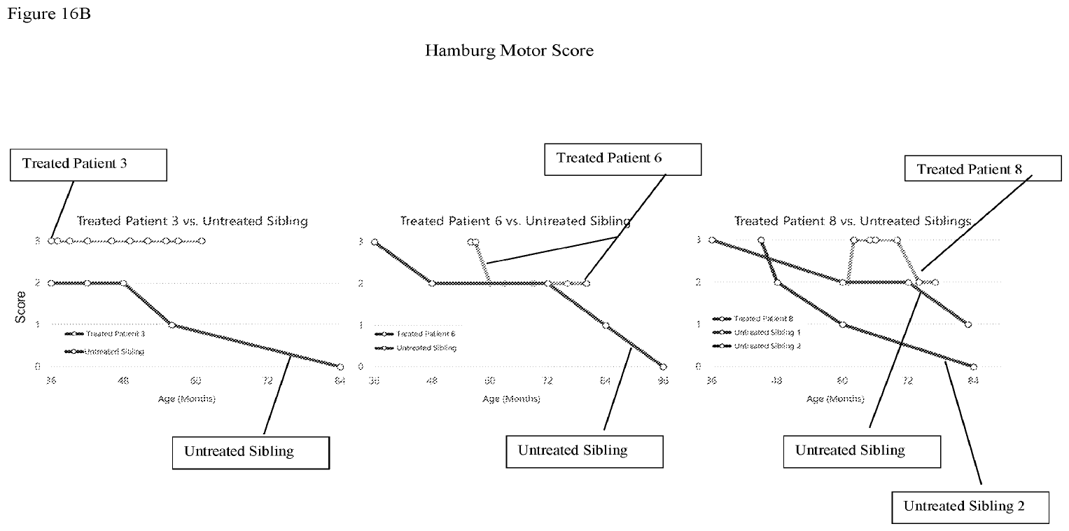

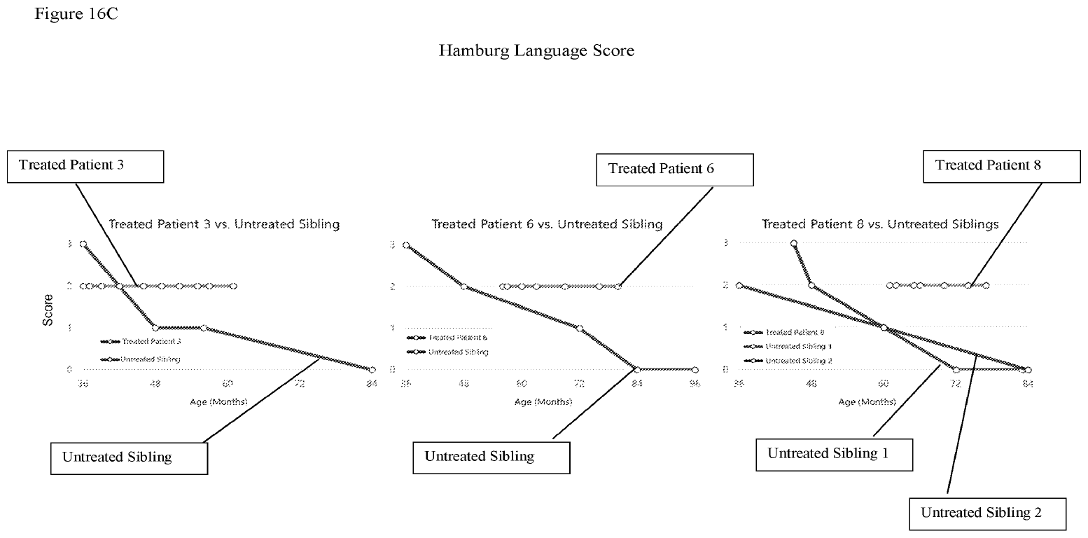

FIG. 16A-C provide sibling comparison data all of whom have CLN6 disease. Treated patients demonstrated stabilization relative to untreated siblings who experienced substantial declines in motor and language ability or died over the same time period. These data are provided as Hamburg Score: Motor+Language over time. FIG. 16A provides the Aggregate scores, while FIG. 16B provides the Hamburg Motor Subscore and FIG. 16C provides the Hamburg Language Subscore.

FIG. 12A-C provide in-study sibling comparison data for in-study pairs who were both treated with scAAV9.CB.CLN6. These data indicated that younger siblings demonstrated an increase or stabilization in Hamburg Motor and Language scores compared to older siblings who had an initial change followed by stabilization. FIG. 12A provides the aggregate scores, while FIG. 12B provides the Hamburg Motor Subscore and FIG. 12 C provides the Hamburg Language Subscore.

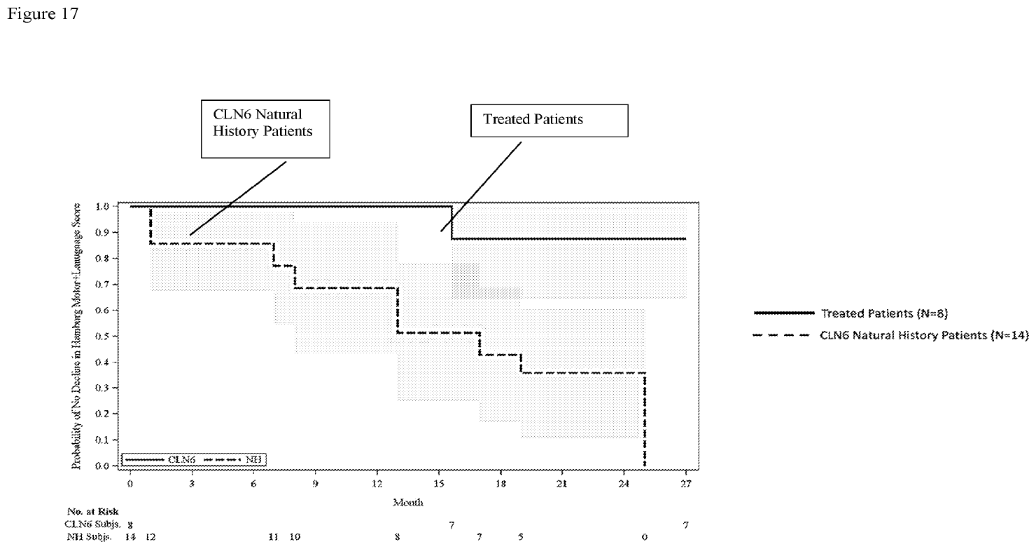

FIG. 17 compares the data for the first 8 patients treated scAAV9.CB.CLN6 to data from an ongoing natural history study of CLN6 patients conducted by Nationwide Children's Hospital (n=14). Shown is a Kaplan-Meier curve for the time until unreversed decrease from baseline of 2 or more points in the combined score for Hamburg Motor and Language function. Confidence bands are calculated using the survival probability estimates and their standard error. The figure compares the patients with ≥2 point decline in the combined Hamburg Motor and Language score in the treated group vs natural history group over a 2 year period and conveys results supporting efficacy of the therapy of the present invention, including: (i) only 1 treated patient achieved a ≥2-point decline over that period compared to all 14 natural history untreated patients; and (ii) a substantial separation of the treated patients from those who are untreated.

In summary, the 24-month efficacy data demonstrated the following: i) stabilization of disease, in contrast to untreated siblings who experienced rapid decline in their motor and language ability, ii) younger patients showed an increase in score or stabilization, and iii) the majority of older patients showed initial change followed by stabilization. In addition, the treatment was generally well tolerated.

### Dose Escalation Study

If there are no safety concerns, after the first cohort is evaluated at one-month post-injection additional subjects will be enrolled. Each subject in cohort 2 (n=4) receives an escalated dose of viral vector. There is at least a six-week window between the completion of Cohort 1 and the start of Cohort 2, to allow a review of the safety analysis from five time points (days 1, 2, 7, 14, and 21) as well as DSMB review prior to dosing of the next subject.

Disease progression is measured with the UBDRS scales or the Hamburg Motor and Language Scale (referenced in the Detailed Description above) and the impact of treatment on quality of life using the Pediatric Quality of Life (PEDSQOL) scale, and potential for prolonged survival.

The primary analysis for efficacy is assessed when all patients have completed the three-year study. Basis of determining efficacy is by stabilization or reduced progression of the disease based on the well-established Unified Batten Disease Rating Scale (UBDRS) that was developed specifically for CLN6-Batten Disease or the Hamburg Motor and Language Scale. Upon completion of the three-year study period, patients will be monitored annually for 5 years as per FDA guidance.

### Example 5

### Natural History Study Demonstrates scAAV9.CB.CLN6 Gene Therapy Improves Motor and Language Scores

To facilitate comparison of study subjects (first patients (n=8) in the CLN6 gene transfer study) to natural history subjects with respect to clinical course over time, combined Hamburg Scale Motor (M) and Language (L) scores from gene transfer patients were matched with the combined Hamburg Scale Motor and Language scores data collected on patients in a retrospective CLN6 natural history study (PI: Emily de los Reyes, MD; ClinicalTrials.gov Identifier: NCT03285425). Gene transfer patients were matched with natural history patients on the basis of baseline Hamburg Motor and Language scores and age at the time of comparison (within 12 months).

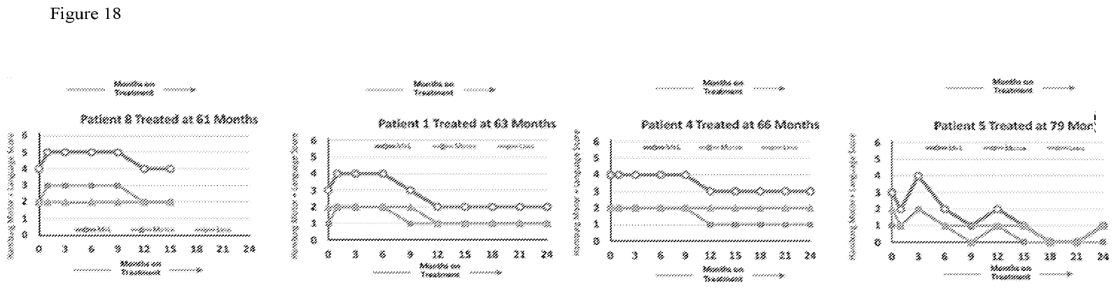

The data for combined and individual data for Hamburg Motor and Language scores (n=8) shows that CLN6 gene therapy halts or substantially slows progression of disease with a positive impact on motor and language function in 7 out of 8 patients (FIG. 18). A positive impact refers to patients that maintained the combined Hamburg score or had an initial change (+1 to −1 points) followed by stabilization. Separate motor and language scores were consistent with the respective combined score.

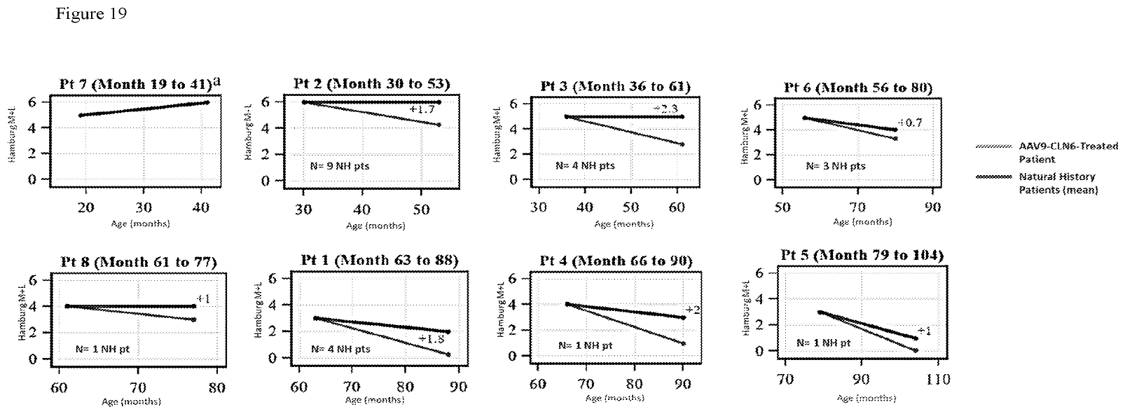

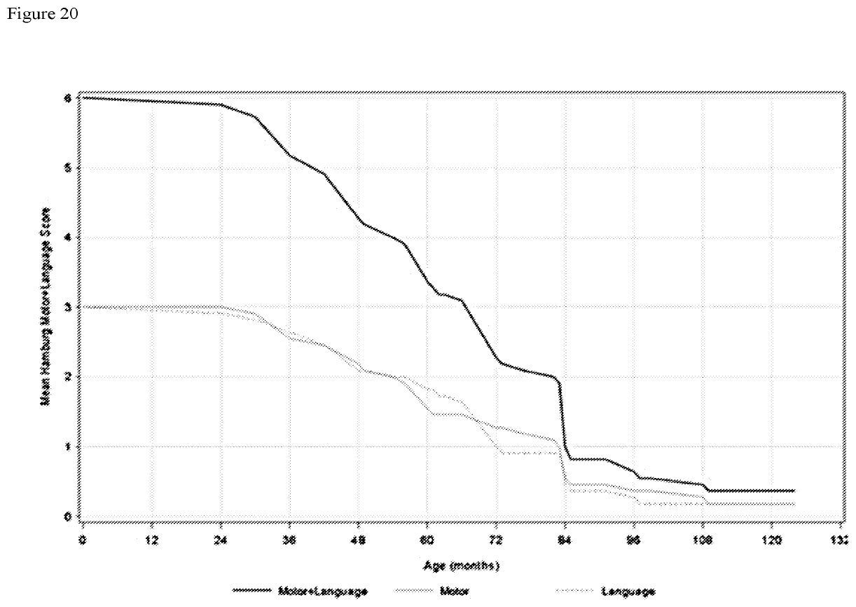

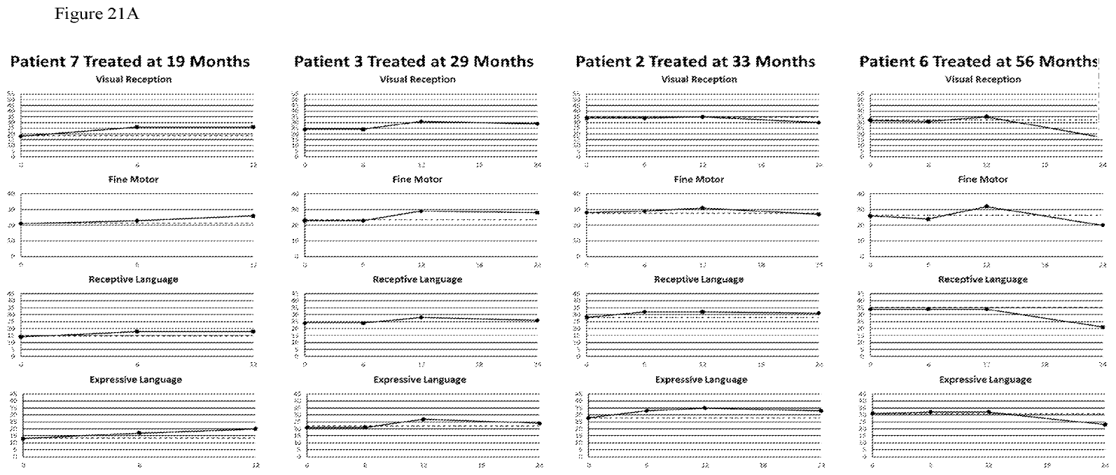

The data for natural history matched comparisons also shows improvement in Hamburg Motor and Language scores (FIG. 19). Via a many-to-one matching methodology, the mean Hamburg Motor and Language scores of the matched natural history patients at the last time point of the comparison period (to the respective gene transfer patient) are plotted in red vs. the respective Hamburg motor and language value of the gene transfer patient at the last time point (plotted in green) (FIG. 19). The number of natural history patients in each comparison are provided in each figure along with the difference between the Hamburg motor and language score at the last time point (between the gene transfer patient and the mean value of the NH patient). Natural history data collected for CLN6-Batten Disease patients (n=11) in a study by Nationwide Children's Hospital and Dr. Emily de los Reyes indicates that there is a fairly linear and almost sustained one-point decline in Hamburg Motor+Language score per year from age two to seven (FIG. 20).

Overall, data from these studies indicate that the majority of CLN6 gene transfer patients demonstrate improvement in motor and language scores compared to matched natural history patients.

### Example 6

### Mullen Scale of Early Learning Analysis

The Mullen Scales of Early Learning (MSEL) were used to evaluate whether scAAV.CB.CLN6 gene therapy improved patients' ability to learn over 12 to 24 months. The MSEL is an individually administered, standardized measure of cognitive functioning designed to be used in children from birth to 68 months. The subscales of the MSEL are gross-motor, fine-motor, receptive language, expressive language, and early learning composite. (See Mullen E M. (1995). Mullen Scales of Early Learning (AGS ed.). Circle Pines, Minn.: American Guidance Service Inc).

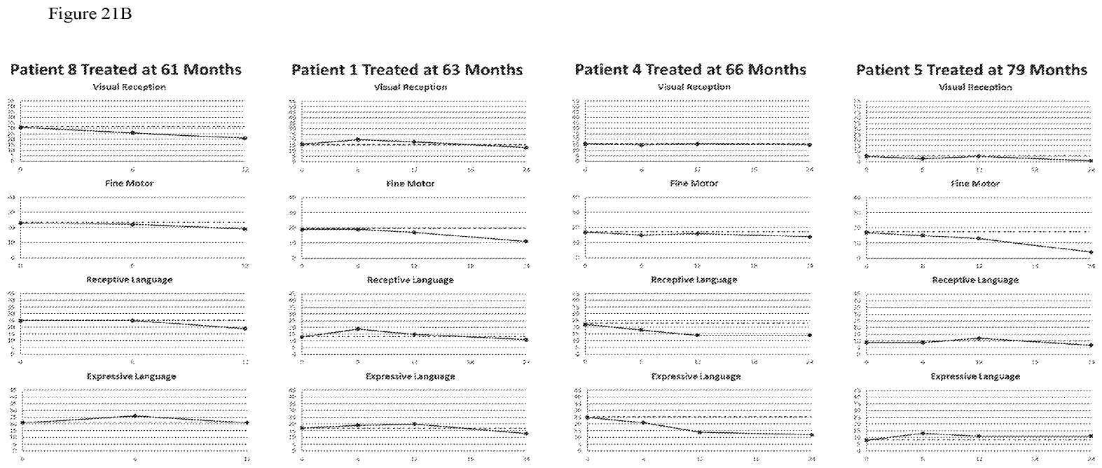

The following 4 domains were analyzed in 8 patients: visual reception, fine motor, receptive language, and expressive language. FIGS. 21A and 21B provide the raw scores for the 4 domains. Higher scores indicate higher function.

### SUMMARY

The interim safety and efficacy data suggest that AAV9-CLN6 gene therapy has the potential to stabilize progression of the variant late-infantile onset CLN6 Batten disease. Efficacy results demonstrated a meaningful treatment effect in motor and language function. AAV9-CLN6-treated patients demonstrated improvement in Hamburg Motor and Language scores compared with untreated siblings and mean values of natural history patients matched for age and Hamburg Motor and Language baseline score. Comparison of treated younger and older siblings further supports the potential benefit of early intervention of gene therapy with AAV9-CLN6. Younger treated patients demonstrated improvement or stabilization in cognitive skills as shown with the MSEL scale.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

What is claimed:

1. A self-complementary recombinant adeno-associated virus 9 (scAAV9) encoding the CLN6 polypeptide, comprising an scAAV9 genome comprising in 5′ to 3′ order: a first AAV inverted terminal repeat, a CB promoter comprising the nucleotide sequence of SEQ ID NO: 3, a polynucleotide encoding the CLN6 polypeptide of SEQ ID NO: 1 and a second AAV inverted terminal repeat.

2. The scAAV9 of claim 1 wherein the scAAV9 genome comprises in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat.

3. The scAAV9 of claim 1 wherein the scAAV9 genome comprises in 5′ to 3′ order: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat.

4. The scAAV9 of any one of claims 1 to 3, wherein the polynucleotide encoding the CLN6 polypeptide comprises a sequence at least 90% identical to SEQ ID NO: 2.

5. The scAAV9 of any one of claims 1 to 3, wherein the polynucleotide encoding the CLN6 polypeptide comprises the nucleic acid sequence of SEQ ID NO: 2.

6. The scAAV9 of any one of claims 1 to 5, wherein the is scAAV9 genome comprises a nucleic acid sequence at least 90% identical to the nucleic acid sequence of SEQ ID NO: 4

7. The scAAV9 of any one of claims 1 to 5, wherein the is scAAV9 genome comprises a nucleic acid sequence at least 95% identical to the nucleic acid sequence of SEQ ID NO: 4.

8. The scAAV9 of any one of claims 1 to 6, wherein the is scAAV9 genome comprises the nucleic acid sequence of SEQ ID NO: 4.

9. The scAAV9 of any one of claims 1 to 8, wherein the AAV inverted terminal repeats are AAV2 inverted terminal repeats.

10. The scAAV9 of any one of claims 1 to 10, wherein the rAAV9 genome comprises a single-stranded genome.

11. A nucleic acid molecule comprising a first AAV inverted terminal repeat, a CB promoter comprising the sequence of SEQ ID NO: 3, a nucleic acid sequence encoding the CLN6 polypeptide of SEQ ID NO: 1 and a second AAV inverted terminal repeat.

12. The nucleic acid molecule of claim 11, comprising a first AAV inverted terminal repeat, a CB promoter comprising the nucleotide sequence of SEQ ID NO: 3, an SV40 intron, a nucleic acid sequence encoding the CLN6 polypeptide of SEQ ID NO: 1 and a second AAV inverted terminal repeat.

13. The nucleic acid molecule of claim 11, comprising a first AAV inverted terminal repeat, a CB promoter comprising the nucleotide sequence of SEQ ID NO: 3, a nucleic acid encoding the CLN6 polypeptide of SEQ ID NO: 1, a bovine growth hormone polyadenylation poly A sequence and a second AAV inverted terminal repeat.

14. The nucleic acid molecule of any one of claims 11 to 13, wherein the nucleic acid encoding the CLN6 polypeptide comprises a sequence at least 90% identical to the nucleic acid sequence of SEQ ID NO: 2.

15. The nucleic acid molecule of any one of claims 11 to 13, wherein the nucleic acid encoding the CLN6 polypeptide comprises the nucleic acid sequence of SEQ ID NO: 2.

16. The nucleic acid molecule of any one of claims 11 to 15, comprising a nucleic acid sequence at least 90% identical to the nucleic acid sequence SEQ ID NO: 4.

17. The nucleic acid molecule of any one of claims 11 to 15 comprising a nucleic acid sequence at least 95% identical to the nucleic acid sequence SEQ ID NO: 4.

18. The nucleic acid molecule of any one of claims 11 to 15 comprising a nucleic acid sequence of SEQ ID NO: 4.

19. The nucleic acid molecule of any one of claims 11 to 18, wherein the AAV inverted terminal repeats are AAV2 inverted terminal repeats.

20. A self-complementary recombinant adeno-associated virus 9 (scAAV9) comprising a nucleic acid molecule of any one of claims 11 to 19.

21. The scAAV9 of claim 20, wherein the scAAV9 comprises a single-stranded genome.

22. An rAAV particle comprising a polynucleotide sequence of any one of claims 11 to 19.

23. The rAAV particle of claim 22 wherein the rAAV particle comprises a single-stranded genome.

24. A recombinant adeno-associated virus 9 (rAAV9) viral particle encoding a CLN6 polypeptide, comprising an rAAV9 genome comprising in 5′ to 3′ order: a CMV enhancer comprising a nucleic acid sequence at least 90% identical to the nucleic acid sequence SEQ ID NO: 6, a chicken 3-actin promoter comprising a nucleic acid sequence at least 90% identical to the nucleic acid sequence SEQ ID NO: 3, and a polynucleotide encoding a CLN6 polypeptide at least 90% identical to the amino acid sequence of SEQ ID NO: 1.

25. The rAAV9 viral particle of claim 24, wherein the rAAV9 genome comprises a self-complementary genome.

26. The rAAV9 viral particle of claim 24, wherein the rAAV9 genome comprises a single-stranded genome.

27. The rAAV9 viral particle of any one of claims 24 to 26, wherein the rAAV9 genome comprises in 5′ to 3′ order: a first AAV inverted terminal repeat, the CMV enhancer comprising a nucleic acid sequence at least 90% identical to the nucleic acid sequence SEQ ID NO: 6, the chicken β-actin promoter comprising a nucleic acid sequence at least 90% identical to the nucleic acid sequence SEQ ID NO: 3, the polynucleotide encoding a CLN6 polypeptide at least 90% identical to the amino acid sequence of SEQ ID NO: 1, and a second AAV inverted terminal repeat.

28. The rAAV9 viral particle of any one of claims 24 to 27, wherein the polynucleotide encoding the CLN6 polypeptide comprises a nucleic acid sequence at least 90% identical to nucleic acid sequence of SEQ ID NO: 2.

29. The rAAV9 viral particle of any one of claims 24 to 28, wherein the rAAV9 genome comprises a nucleic acid sequence at least 90% identical to the nucleic acid sequence SEQ ID NO: 4.

30. The rAAV9 viral particle of any one of claims 24 to 28, wherein the rAAV9 genome comprises a nucleic acid sequence at least 95% identical to the nucleic acid sequence SEQ ID NO: 4.

31. The rAAV9 viral particle of any one of claims 24 to 30, wherein the AAV inverted terminal repeats are AAV2 inverted terminal repeats.

32. The rAAV9 viral particle of any one of claims 24 to 31, wherein the rAAV9 genome further comprises an SV40 intron.

33. The rAAV9 viral particle of any one of claims 24 to 32, wherein the rAAV9 genome further comprises a BGH poly-A sequence.

34. A nucleic acid molecule comprising an rAAV9 genome comprising in 5′ to 3′ order: a first AAV inverted terminal repeat, a CMV enhancer having a nucleic acid sequence at least 90% identical to the nucleic acid sequence of SEQ ID NO: 6, a chicken 3-actin promoter having a nucleic acid sequence at least 90% identical to the nucleic acid sequence of SEQ ID NO: 3, a polynucleotide encoding a CLN6 polypeptide at least 90% identical to the amino acid sequence of SEQ ID NO: 1, and a second AAV inverted terminal repeat.

35. The nucleic acid molecule of claim 34, herein the rAAV9 genome comprises a self-complementary genome.

36. The nucleic acid molecule of claim 34, wherein the rAAV9 genome comprises a single-stranded genome.

37. The nucleic acid molecule of any one of claims 34 to 36, wherein the rAAV9 genome comprises in 5′ to 3′ order: a first AAV inverted terminal repeat, the CMV enhancer having a nucleic acid sequence at least 90% identical to the nucleic acid sequence of SEQ ID NO: 6, the chicken 3-actin promoter having a nucleic acid sequence at least 90% identical to the nucleic acid sequence of SEQ ID NO: 3, the polynucleotide encoding a CLN6 polypeptide at least 90% identical to the amino acid sequence of SEQ ID NO: 1, and a second AAV inverted terminal repeat.

38. The nucleic acid molecule of any one of claims 34 to 37, wherein the polynucleotide encoding the CLN6 polypeptide comprises an amino acid sequence at least 90% identical to the nucleic acid sequence of SEQ ID NO: 2.

39. The nucleic acid molecule of any one of claims 34 to 38, wherein the rAAV9 genome comprises an amino acid sequence at least 90% identical to SEQ ID NO: 4.

40. The nucleic acid molecule of any one of claims 34 to 38, wherein the rAAV9 genome comprises a sequence at least 95% identical to SEQ ID NO: 4.

41. The nucleic acid molecule of any one of claims 34 to 40, wherein the AAV inverted terminal repeats are AAV2 inverted terminal repeats.

42. The nucleic acid molecule of any one of claims 34 to 41, wherein the rAAV9 genome further comprises an SV40 intron.

43. The nucleic acid molecule of any one of claims 34 to 42, wherein the rAAV9 genome further comprises a BGH poly-A sequence.

44. A composition comprising a scAAV9 of any one of claims 1 to 10, the nucleic acid molecule of any one of claims 11 to 19, 31 or 34 to 43, the rAAV9 viral particle of any one of claims 22 to 33 and a pharmaceutically acceptable excipient, carrier, or diluent.

45. The composition of claim 44, wherein the excipient comprises a non-ionic, low-osmolar compound, a buffer, a polymer, a salt, or a combination thereof.

46. A method of treating CLN6-Batten Disease in an subject comprising administering to the subject a composition comprising a therapeutically effective amount of the scAAV9 of any one of claims 1 to 10, the rAAV9 viral particle of any one of claims 22 to 33, the nucleic acid of any one of claims 11 to 19, 31 or 34 to 43, or the composition of claim 44 or claim 45.

47. The method of claim 46, wherein the composition is administered via a route selected from the group consisting of intrathecal, intracerebroventricular, intraparenchymal, intravenous, and a combination thereof.

48. The method of claim 47, wherein the composition is administered intrathecally.

49. The method of claim 47, wherein the composition is administered intracerebroventricularly.

50. The method of claim 4746, wherein the composition is administered intravenously.

51. The method of any one of claims 46 to 50, wherein about 1×10¹¹ to about 1×10¹⁵ vg of the rAAV9 viral particle is administered.

52. The method of any one of claims 46 to 51, wherein about 1×10¹² to about 1×10¹⁴ of the rAAV9 viral particle is administered.

53. The method of any one of claims 46 to 52, wherein the treatment stabilizes or slows one or more symptoms of CLN-6 Batten Disease selected from:
(a) loss of brain volume;
(b) loss of cognitive function; and
(c) language delay;
as compared to an untreated CLN6-Batten Disease patient.

54. The method of any one of claims 46 to 52, wherein the treatment stabilizes or slows disease progression of CLN-6 Batten Disease.

55. The method of claim 54, wherein disease progression is assessed with the UBDRS scales, the Hamburg Motor and Language Scale, the impact of treatment on quality of life using the Pediatric Quality of Life (PEDSQOL) scale, the Mullen Scales of Early Learning (MSEL), the potential for prolonged survival, or a combination thereof.

56. The method of any one of claims 46 to 55, wherein the subject is aged 80 months or under, 75 months or under, 70 months or under, 65 months or under, 62 months or under, 60 months or under, 55 months or under, 50 months or under, or 40 months or under.

57. The method of any one of claims 46 to 56, further comprising placing the subject in the Trendelenberg position after administering the rAAV9 viral particle.

58. A method of treating a CLN6 disease in a patient in need thereof comprising, delivering a composition comprising the scAAV of any one of claims 1 to 10, the rAAV9 viral particle of any one of claims 22 to 33, the nucleic acid of any one of claims 11 to 19, 31 or 34 to 4334, or the composition of claim 44 or claim 45 to a brain or spinal cord of a patient in need thereof.

59. The method of claim 55, wherein the composition is delivered by intrathecal, intracerebroventricular, intraparenchymal, or intravenous injection, or a combination thereof.

60. The method of claim 56, further comprising placing the patient in the Trendelenberg position after intrathecal injection of the composition.

61. The method of any one of claims to 55 to 57, wherein the composition comprises a non-ionic, low-osmolar contrast agent.

62. The method of claim 5861, wherein the non-ionic, low-osmolar contrast agent is selected from the group consisting of iobitridol, iohexol, iomeprol, iopamidol, iopentol, iopromide, ioversol, ioxilan, and combinations thereof.

63. The method of any one of claims 55 to 59, wherein the delivering to the brain or spinal cord comprises delivery to a brain stem.

64. The method of any one of claims 55 to 59, wherein the delivering to the brain or spinal cord comprises delivery to a cerebellum.

65. The method of any one of claims 55 to 59, wherein the delivering to the brain or spinal cord comprises delivery to a visual cortex.

66. The method of any one of claims 55 to 59, wherein the delivering to the brain or spinal cord comprises delivery to a motor cortex.

67. The method of any one of claims 55 to 63, wherein the delivering to the brain or spinal cord comprises delivery to a nerve cell, a glial cell, or both.

68. The method of any one of claims 55 to 63, wherein the delivering to the brain or spinal cord comprises delivery to cell of the nervous system, wherein the cell of the nervous system is a neuron, a lower motor neuron, a microglial cell, an oligodendrocyte, an astrocyte, a Schwann cell, or a combination thereof.

69. The method of any one of claims 58 to 68, wherein the treatment stabilizes or slows one or more symptoms of CLN-6 Batten Disease selected from:
(a) loss of brain volume;
(b) loss of cognitive function; and
(c) language delay;
as compared to an untreated CLN6-Batten Disease patient.

70. The method of any one of claims 58 to 68, wherein the treatment stabilizes or slows disease progression of CLN-6 Batten Disease.

71. The method of claim 70, wherein disease progression is assessed with the UBDRS scales, the Hamburg Motor and Language Scale, the impact of treatment on quality of life using the Pediatric Quality of Life (PEDSQOL) scale, the Mullen Scales of Early Learning (MSEL), the potential for prolonged survival, or a combination thereof.

72. The method of any one of claims 58 to 71, wherein the patient is aged 80 months or under, 75 months or under, 70 months or under, 65 months or under, 62 months or under, 60 months or under, 55 months or under, 50 months or under, or 40 months or under.

73. Use of a therapeutically effective amount of the scAAV9 of any one of claims 1 to 10, the rAAV9 viral particle of any one of claims 22 to 33, the nucleic acid of any one of claims 11 to 19, 31 or 34 to 43, or the composition of claim 44 or claim 45 for the preparation of a medicament for treating CLN6-Batten Disease in an subject.

74. A composition comprising a therapeutically effective amount of the scAAV9 of any one of claims 1 to 10, the rAAV9 viral particle of any one of claims 22 to 33, the nucleic acid of any one of claims 11 to 19, 31 or 34 to 43, or the composition of claim 44 or claim 45 for treating CLN6-Batten Disease in an subject.

75. Use of a composition comprising the scAAV of any one of claims 1 to 10, the rAAV9 viral particle of any one of claims 22 to 33, the nucleic acid of any one of claims 11 to 19, 31 or 34 to 4334, or the composition of claim 44 or claim 45 for the preparation of a medicament for delivering said rAAV viral particle, nucleic acid, or composition to a brain or spinal cord of a patient in need thereof.

76. A composition for treating a CLN6 disease in a patient in need thereof, wherein the composition comprises the scAAV of any one of claims 1 to 10, the rAAV9 viral particle of any one of claims 22 to 33, the nucleic acid of any one of claims 11 to 19, 31 or 34 to 4334, or the composition of claim 44 or claim 45 to a brain or spinal cord of a patient in need thereof.


SEQUENCE LISTING (11 sequences)

SEQ ID NO: 1 - protein, 311 aa, Homo sapiens
  MISC_FEATURE: CLN6 Polypeptide Sequence
MEATRRRQHLGATGGPGAQLGASFLQARHGSVSADEAARTAPFHLDLWFYFTLQNWVLDFGRPIAMLVFPLEWFPLNKPSVGDYFHMAYNVITPFLLLKLIERSPRTLPRSITYVSIIIFIMGASIHLVGDSVNHRLLFSGYQHHLSVRENPIIKNLKPETLIDSFELLYYYDEYLGHCMWYIPFFLILFMYFSGCFTASKAESLIPGPALLLVAPSGLYYWYLVTEGQIFILFIFTFFAMLALVLHQKRKRLFLDSNGLFLFSSFALTLLLVALWVAWLWNDPVLRKKYPGVIYVPEPWAFYTLHVSSRH

SEQ ID NO: 2 - DNA, 936 bp, Homo sapiens
  misc_feature: CLN6  polynucleotide sequence
atggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactga

SEQ ID NO: 3 - DNA, 270 bp, Artificial Sequence
  Synthetic Polynucleotide
  misc_feature: hybrid chicken B -Actin promoter
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 4 - DNA, 2343 bp, Artificial Sequence
  Synthetic Polynucleotide
  misc_feature: Gene Cassette
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactgagtctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 5 - DNA, 6028 bp, Artificial Sequence
  Synthetic Polynucleotide
  misc_feature: AAV.CB.CLN6
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactgagtctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 6 - DNA, 280 bp, Artificial Sequence
  Synthetic Polynucleotide
  misc_feature: CMV Sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 7 - protein, 736 aa, Artificial Sequence
  Synthetic Polypeptide
  MISC_FEATURE: scAAV9 seuqence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 141 bp, Artificial Sequence
  Synthetic Polynucleotide
  misc_feature: AAV2 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 9 - DNA, 106 bp, Artificial Sequence
  Synthetic Polynucleotide
  misc_feature: AAV2 5′ ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 10 - DNA, 147 bp, Artificial Sequence
  Synthetic Polyucleotide
  misc_feature: BGH Poly A
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 11 - DNA, 60 bp, Artificial Sequence
  Synthetic Polynucleotide
  misc_feature: SV40 intron
gtaagtttagtctttttgtcttttatttcaggtcccggatccggtggtggtgcaaatcaa